(19) United States
(12) Patent Application Publication
(10) Pub. No.: US 2022/0298564 A1
(43) Pub. Date: Sep. 22, 2022

(54) HIGH SENSITIVITY DETECTION OF MICROSATELLITE LOCI BY BLOCKER DISPLACEMENT AMPLIFICATION WITH MULTIPLE BLOCKERS AND ITS USE IN MICROSATELLITE INSTABILITY DETECTION

(71) Applicant: NuProbe USA, Inc., Houston, TX (US)

(72) Inventor: Yan YAN, Houston, TX (US)

(73) Assignee: NuProbe USA, Inc., Houston, TX (US)

(21) Appl. No.: 17/499,536

(22) Filed: Oct. 12, 2021

Related U.S. Application Data
(60) Provisional application No. 63/161,766, filed on Mar. 16, 2021.

(51) Int. Cl. C12Q 1/6853; C12Q 1/6876

(52) U.S. Cl. CPC C12Q 1/6853; C12Q 1/6876

(57) ABSTRACT

This invention describes methods and compositions for selectively enriching unstable variants at a particular microsatellite locus.

### CROSS-REFERENCE TO RELATED APPLICATION

This application claims benefit of U.S. Provisional Application No. 63/161,766, which was filed on Mar. 16, 2021, the entire contents of which is incorporated herein by reference.

### FIELD

The present disclosure relates to the field of molecular biology. More particularly, it relates to methods and compositions useful for the amplification of nucleic acid molecules.

### INCORPORATION OF SEQUENCE LISTING

A sequence listing contained in the file named “P35059US01_SL.txt” which is 16,384 bytes (measured in MS-Windows®) and created on Oct. 12, 2021, is filed electronically herewith and incorporated by reference in its entirety.

### BACKGROUND

Microsatellite instability (MSI) refers to genetic instability in short nucleotide repeats (e.g., microsatellites), where a cell comprises a different number of repeats as compared to what was inherited from a progenitor cell. This genetic instability is often caused by high mutation rates resulting from abnormal DNA mismatch repair, especially in some types of cancers. Capillary electrophoresis is often used to detect microsatellite instability, however, it has no better than 5% analytical sensitivity for any given microsatellite instability locus.

Microsatellite instability is a guideline-recommended biomarker used in assessment of prognosis and treatment choices, including checkpoint inhibitors recently approved for the treatment of cancers with MSI high (MSI-H) status. Plasma-based next generation DNA sequencing (NGS) tests are increasingly used for comprehensive genomic profiling of cancer, however, methods to detect MSI status from cell-free DNA (cfDNA) data are underdeveloped. There is a need in the art to improve the sensitivity of detecting microsatellite instability. Here, a novel methods and compositions are provided to reduce the analytical sensitivity of a microsatellite instability locus to at least 0.1%.

### SUMMARY

In one aspect, this disclosure provides a composition comprising: (a) a DNA template molecule comprising, continuously from 5′ to 3′ (i) an upstream sequence to a microsatellite repetitive sequence; (ii) the microsatellite repetitive sequence; and (iii) a downstream sequence to the microsatellite repetitive sequence; and (b) a plurality of non-extensible oligonucleotides, where each of the plurality of non-extensible oligonucleotides comprises, from 5′ to 3′, (i) a first binding sequence that is identical to the reverse complement of at least part of the upstream sequence; (ii) a second binding sequence that is identical to the reverse complement of the microsatellite repetitive sequence or a variant thereof; (iii) a third binding sequence that is identical to the reverse complement of at least part of the downstream sequence; and (iv) a terminator sequence comprising only adenine or only thymine nucleotides, and where the terminator sequence is not identical to the reverse complement of the at least part of the downstream sequence.

In one aspect, this disclosure provides a composition comprising a plurality of non-extensible oligonucleotides, where each of the at least two non-extensible nucleotides comprises, from 5′ to 3′, (i) a first binding sequence that is identical to the reverse complement of at least part of an upstream sequence of a DNA template molecule; (ii) a second binding sequence that is identical to the reverse complement of a microsatellite repetitive sequence or a variant thereof, where the microsatellite repetitive sequence is positioned 3′ to the upstream sequence; (iii) a third binding sequence that is identical to the reverse complement of at least part of a downstream sequence of the DNA template molecule, where the downstream sequence is positioned 3′ to the microsatellite repetitive sequence; and (iv) a terminator sequence comprising only adenine or only thymine nucleotides, and where the terminator sequence is not identical to the reverse complement of the at least part of the downstream sequence.

In one aspect, this disclosure provides a method for selectively inhibiting a polymerase chain reaction amplification of at least one DNA template molecule comprising at least one microsatellite repetitive sequence, the method comprising: (a) preparing a mixture comprising: (i) a plurality of non-extensible oligonucleotides, where each of the at least two non-extensible nucleotides comprises, from 5′ to 3′, (A) a first binding sequence that is identical to the reverse complement of at least part of an upstream sequence of the at least one DNA template molecule; (B) a second binding sequence that is identical to the reverse complement of a microsatellite repetitive sequence or a variant thereof, where the microsatellite repetitive sequence is positioned 3′ to the upstream sequence; (C) a third binding sequence that is identical to the reverse complement of at least part of a downstream sequence of the at least one DNA template molecule, where the downstream sequence is positioned 3′ to the microsatellite repetitive sequence; and (D) a terminator sequence comprising only adenine or only thymine nucleotides, and where the terminator sequence is not identical to the reverse complement of the at least part of the downstream sequence; (ii) the at least one DNA template molecule; (iii) a DNA polymerase; (iv) dNTPs; (v) a forward primer and a reverse primer, where the forward and reverse primer are capable of amplifying the at least one DNA template molecule; and (b) subjecting the mixture to at least seven cycles of thermal cycling to produce at least one amplicon of at least one member of the group of DNA template molecules.

In one aspect, this disclosure provides a kit comprising: (a) a plurality of non-extensible oligonucleotides, where each of the plurality of non-extensible nucleotides comprises, from 5′ to 3′, (i) a first binding sequence that is identical to the reverse complement of at least part of an upstream sequence of a DNA template molecule; (ii) a second binding sequence that is identical to the reverse complement of a microsatellite repetitive sequence or a variant thereof, where the microsatellite repetitive sequence is positioned 3′ to the upstream sequence; (iii) a third binding sequence that is identical to the reverse complement of at least part of a downstream sequence of the DNA template molecule, where the downstream sequence is positioned 3′ to the microsatellite repetitive sequence; and (iv) a terminator sequence comprising only adenine or only thymine nucleotides, and where the terminator sequence is not identical to the reverse complement of the at least part of the downstream sequence; and (b) a forward primer and a reverse primer, where the forward and reverse primer are capable of amplifying the DNA template molecule.

In an aspect, this disclosure provides a method of determining the instability status of a sample comprising: (a) preparing a mixture comprising: (i) a plurality of non-extensible oligonucleotides, where each of the at least two non-extensible nucleotides comprises, from 5′ to 3′, (A) a first binding sequence that is identical to the reverse complement of at least part of an upstream sequence of a DNA template molecule; (B) a second binding sequence that is identical to the reverse complement of a microsatellite repetitive sequence or a variant thereof, where the microsatellite repetitive sequence is positioned 3′ to the upstream sequence; (C) a third binding sequence that is identical to the reverse complement of at least part of a downstream sequence of the DNA template molecule, where the downstream sequence is positioned 3′ to the microsatellite repetitive sequence; and (D) a terminator sequence comprising only adenine or only thymine nucleotides, and where the terminator sequence is not identical to the reverse complement of the at least part of the downstream sequence, where the plurality of non-extensible oligonucleotides target at least two microsatellite loci; (ii) the DNA template molecule, where the DNA template molecule is obtained from the sample; (iii) a DNA polymerase; (iv) dNTPs; (v) at least two primer sets, where the at least two primer sets are capable of amplifying the at least two microsatellite loci; (b) subjecting the mixture to at least seven cycles of thermal cycling to produce at least one amplicon of each of the at least two microsatellite loci; and (c) determining the instability status of the sample based on analysis of the at least one amplicon of each of the at least two microsatellite loci obtained in step (b).

### BRIEF DESCRIPTION OF THE DRAWINGS

FIG. 1 depicts components of the non-extensible oligonucleotides provided herein. Region 1 refers to an upstream sequence. Region 2 refers to a microsatellite repetitive sequence (here, a homopolymer). Region 3 refers to a downstream sequence. Region 4 refers to a terminator sequence. Regions 1, 3, and 4 are shared amongst each individual non-extensible oligonucleotide (e.g., T(24), T(23), T(22), T(21), T(20), T(19), T(18)). However, Region 2 differs in each non-extensible oligonucleotide. Only the T(21) non-extensible oligonucleotide is a perfect match to the NR21 DNA template sequence reverse complement.

FIG. 2 depicts the detection and design from the reverse complement DNA template of FIG. 1. The NR21 microsatellite locus in gene SLC7A8 comprises a homopolymer tract of 21 adenines.

FIG. 3 depicts an embodiment using multiple non-extensible oligonucleotides. Depending on the distribution of microsatellite repeat sizes in a DNA template, non-extensible oligonucleotides having different microsatellite repeat numbers (e.g., Region 2 in FIG. 1) can be applied. The number of repeats in the DNA template sequence is denoted as (n). Blockers targeting (n−y) to (n+x) can be applied. X and y can be equal or non-equal.

FIG. 4 depicts experimental demonstration of blocker displacement amplification using non-extensible oligonucleotides (e.g., blockers). The upper panel depicts cycle threshold (Ct) values, while the lower panel depicts fluorescence traces used to generate the upper panel.

### DETAILED DESCRIPTION

Unless defined otherwise, all technical and scientific terms used have the same meaning as commonly understood by one of ordinary skill in the art to which this disclosure belongs. Where a term is provided in the singular, the inventors also contemplate aspects of the disclosure described by the plural of that term. Where there are discrepancies in terms and definitions used in references that are incorporated by reference, the terms used in this application shall have the definitions given herein. Other technical terms used have their ordinary meaning in the art in which they are used, as exemplified by various art-specific dictionaries, for example, “The American Heritage® Science Dictionary” (Editors of the American Heritage Dictionaries, 2011, Houghton Mifflin Harcourt, Boston and New York), the “McGraw-Hill Dictionary of Scientific and Technical Terms” (6th edition, 2002, McGraw-Hill, New York), or the “Oxford Dictionary of Biology” (6th edition, 2008, Oxford University Press, Oxford and New York).

Any references cited herein, including, e.g., all patents, published patent applications, and non-patent publications, are incorporated herein by reference in their entirety.

Any composition provided herein is specifically envisioned for use with any applicable method provided herein. Any composition provided herein is specifically envisioned for use in a kit.

When a grouping of alternatives is presented, any and all combinations of the members that make up that grouping of alternatives is specifically envisioned. For example, if an item is selected from a group consisting of A, B, C, and D, the inventors specifically envision each alternative individually (e.g., A alone, B alone, etc.), as well as combinations such as A, B, and D; A and C; B and C; etc.

When a range of numbers is provided herein, the range is understood to inclusive of the edges of the range as well as any number between the defined edges of the range. For example, “between 1 and 10” includes any number between 1 and 10, as well as the number 1 and the number 10.

As used herein, the singular form “a,” “an,” and “the” include plural references unless the context clearly dictates otherwise. For example, the term “a compound” or “at least one compound” may include a plurality of compounds, including mixtures thereof. As used herein, the term “plurality” refers to any whole number greater than one.

In an aspect, a plurality refers to at least 2. In an aspect, a plurality refers to at least 3. In an aspect, a plurality refers to at least 4. In an aspect, a plurality refers to at least 5. In an aspect, a plurality refers to at least 6. In an aspect, a plurality refers to at least 7. In an aspect, a plurality refers to at least 8. In an aspect, a plurality refers to at least 9. In an aspect, a plurality refers to at least 10. In an aspect, a plurality refers to at least 11. In an aspect, a plurality refers to at least 12. In an aspect, a plurality refers to at least 13. In an aspect, a plurality refers to at least 14. In an aspect, a plurality refers to at least 15. In an aspect, a plurality refers to at least 20. In an aspect, a plurality refers to at least 25. In an aspect, a plurality refers to at least 30. In an aspect, a plurality refers to at least 40. In an aspect, a plurality refers to at least 50. In an aspect, a plurality refers to at least 60. In an aspect, a plurality refers to at least 70. In an aspect, a plurality refers to at least 80. In an aspect, a plurality refers to at least 90. In an aspect, a plurality refers to at least 100. In an aspect, a plurality refers to at least 200. In an aspect, a plurality refers to at least 300. In an aspect, a plurality refers to at least 400. In an aspect, a plurality refers to at least 500. In an aspect, a plurality refers to at least 600. In an aspect, a plurality refers to at least 700. In an aspect, a plurality refers to at least 800. In an aspect, a plurality refers to at least 900. In an aspect, a plurality refers to at least 1,000. In an aspect, a plurality refers to at least 1,250. In an aspect, a plurality refers to at least 1,500. In an aspect, a plurality refers to at least 1,750. In an aspect, a plurality refers to at least 2,000. In an aspect, a plurality refers to at least 3,000. In an aspect, a plurality refers to at least 4,000. In an aspect, a plurality refers to at least 5,000.

Methods, kits, and compositions provided herein are useful for improving the sensitivity of detecting microsatellite repetitive sequences using polymerase chain reactions (PCR). Methods, kits, and compositions provided herein can also be useful for determining the MSI status of a subject.

In one aspect, this disclosure provides a composition comprising: (a) a DNA template molecule comprising, continuously from 5′ to 3′ (i) an upstream sequence to a microsatellite repetitive sequence; (ii) the microsatellite repetitive sequence; and (iii) a downstream sequence to the microsatellite repetitive sequence; and (b) a plurality of non-extensible oligonucleotides, where each of the plurality of non-extensible oligonucleotides comprises, from 5′ to 3′, (i) a first binding sequence that is identical to the reverse complement of at least part of the upstream sequence; (ii) a second binding sequence that is identical to the reverse complement of the microsatellite repetitive sequence or a variant thereof; (iii) a third binding sequence that is identical to the reverse complement of at least part of the downstream sequence; and (iv) a terminator sequence comprising only adenine or only thymine nucleotides, and where the terminator sequence is not identical to the reverse complement of the at least part of the downstream sequence.

In one aspect, this disclosure provides a composition comprising a plurality of non-extensible oligonucleotides, where each of the at least two non-extensible nucleotides comprises, from 5′ to 3′, (i) a first binding sequence that is identical to the reverse complement of at least part of an upstream sequence of a DNA template molecule; (ii) a second binding sequence that is identical to the reverse complement of a microsatellite repetitive sequence or a variant thereof, where the microsatellite repetitive sequence is positioned 3′ to the upstream sequence; (iii) a third binding sequence that is identical to the reverse complement of at least part of a downstream sequence of the DNA template molecule, where the downstream sequence is positioned 3′ to the microsatellite repetitive sequence; and (iv) a terminator sequence comprising only adenine or only thymine nucleotides, and where the terminator sequence is not identical to the reverse complement of the at least part of the downstream sequence. In an aspect, the composition further comprises the DNA template molecule.

As used herein, “DNA” or “deoxyribonucleic acid” revers to a natural or modified nucleotide which has a hydrogen group at the 2′-position of the sugar moiety. DNA typically comprises a chain of nucleotides comprising four types of nucleotide bases: adenine (A), guanine (G), thymine (T), and cytosine (C). As is known in the art, certain pairs of nucleotides specifically bind to one another in a complementary fashion (known as complementary base pairing). In DNA, adenine pairs with thymine and cytosine pairs with guanine. In an aspect, a DNA molecule is single-stranded. In an aspect, a DNA molecule is double-stranded. In an aspect, a DNA molecule is both single-stranded and double-stranded.

As used herein, a “DNA template molecule” refers to a DNA molecule that comprises a sequence that is desired to be amplified. In an aspect, a DNA template molecule is a single-stranded DNA molecule. In an aspect, a DNA template molecule is a double-stranded DNA template molecule. In an aspect, a DNA template molecule comprises DNA from a nuclear genome. In an aspect, a DNA template molecule comprises DNA from a mitochondrial genome.

A DNA template molecule can be from any organism. In an aspect, a DNA template molecule is a prokaryotic DNA template molecule. In an aspect, a DNA template molecule is a eukaryotic DNA template molecule. In an aspect, a DNA template molecule is a viral DNA template molecule. In an aspect, a DNA template molecule is a plant DNA template molecule. In an aspect, a DNA template molecule is a fungal DNA template molecule. In an aspect, a DNA template molecule is a protozoan DNA template molecule. In an aspect, a DNA template molecule is an animal DNA template molecule. In an aspect, a DNA template molecule is a mammalian DNA template molecule. In an aspect, a DNA template molecule is a primate DNA template molecule. In an aspect, a DNA template molecule is a human DNA template molecule. In an aspect, a DNA template molecule is a human cancer cell DNA template molecule. In an aspect, a human cancer cell is selected from the group consisting of a colorectal cancer cell, a gastric cancer cell, and an endometrial cancer cell. In an aspect, a human cancer cell is a colorectal cancer cell. In an aspect, a human cancer cell is a gastric cancer cell. In an aspect, a human cancer cell is an endometrial cancer cell.

As used herein, “cancer,” refers to a type or subtype of cancer defined, e.g., by histopathology. Cancer type can be defined by any conventional criterion, such as on the basis of occurrence in a given tissue (e.g., blood cancers, central nervous system (CNS), brain cancers, lung cancers (small cell and non-small cell), skin cancers, nose cancers, throat cancers, liver cancers, bone cancers, lymphomas, pancreatic cancers, bowel cancers, rectal cancers, thyroid cancers, bladder cancers, kidney cancers, mouth cancers, stomach cancers, breast cancers, prostate cancers, ovarian cancers, lung cancers, intestinal cancers, soft tissue cancers, neuroendocrine cancers, gastroesophageal cancers, head and neck cancers, gynecological cancers, colorectal cancers, urothelial cancers, solid state cancers, heterogeneous cancers, homogenous cancers), unknown primary origin and the like, and/or of the same cell lineage (e.g., carcinoma, sarcoma, lymphoma, cholangiocarcinoma, leukemia, mesothelioma, melanoma, or glioblastoma) and/or cancers exhibiting cancer markers, such as, without being limiting, Her2, CA15-3, CA19-9, CA-125, CEA, AFP, PSA, HCG, hormone receptor and NMP-22. Cancers can also be classified by stage (e.g., stage 1, 2, 3, or 4) and whether of primary or secondary origin.

In an aspect, a DNA template molecule is obtained from a sample. As used herein, a “sample” refers to any biological material that is capable of being analyzed by or subjected to the methods, compositions, and/or kits provided herein. Any suitable method known in the art can be used to obtain a DNA template molecule from a sample.

In an aspect, a sample is obtained from a subject. As used herein, a “subject” refers to an animal (e.g., without being limiting, a mammal, reptile, bird, fish, amphibian) or other organism, such as, without being limiting, a plant or fungus. A subject can be a healthy individual, an individual that has or is suspected of having a disease or a predisposition to the disease, or an individual that is in need of therapy or suspected of needing therapy. The term “individual” and “subject” are intended to be interchangeable.

In an aspect, a sample comprises a cell. In an aspect, a sample comprises a tissue. In an aspect, a sample comprises an organ. In an aspect, a sample comprises blood. In an aspect, a sample comprises plasma. In an aspect, a sample comprises urine. In an aspect, a sample comprises feces. In an aspect, a sample comprises cell-free DNA. Additional non-limiting examples of samples include serum, sputum, semen, vaginal fluid, synovial fluid, spinal fluid, and saliva.

As used herein, “cell-free DNA” or “cfDNA” refers to DNA that is not contained within or otherwise bound to a cell. In an aspect, cfDNA refers to DNA that remains following the removal of intact cells. Cell-free DNA can be obtained, without being limiting, from bodily fluids such as blood, plasma, serum, urine, and cerebrospinal fluid. In an aspect, cfDNA is single-stranded. In an aspect, cfDNA is double-stranded. In an aspect, cfDNA comprises both single-stranded and double-stranded DNA. Without being limiting, cfDNA can be released into bodily fluid through secretion or cell death processes (e.g., cellular necrosis, apoptosis). Some cfDNAs are released into bodily fluid from cancer cells (e.g., circulating tumor DNA (ctDNA)). In an aspect, cfDNA comprises ctDNA. Other cfDNAs are released from healthy cells. Without being limiting, ctDNA can be non-encapsulated tumor-derived fragmented DNA. Another example of cfDNA is fetal DNA circulating freely in the maternal blood stream, also called cell-free fetal DNA (cffDNA). In an aspect, cfDNA comprises cffDNA. A cfDNA can have one or more epigenetic modifications, for example and without being limiting, a cfDNA can be acetylated, methylated, ubiquitylated, phosphorylated, sumoylated, and/or ribosylated.

In an aspect, a sample is a eukaryotic sample. In an aspect, a sample is a prokaryotic sample. In an aspect, a sample is a viral sample. In an aspect, a sample is an animal sample. In an aspect, a sample is a plant sample. In an aspect, a sample is a fungal sample. In an aspect, a sample is a mammalian sample. In an aspect, a sample is a primate sample. In an aspect, a sample is a human sample. In an aspect, a sample is a human cancer cell. In an aspect, a human cancer cell is selected from the group consisting of a colorectal cancer cell, a gastric cancer cell, and an endometrial cancer cell. In an aspect, a human cancer cell is a colorectal cancer cell. In an aspect, a human cancer cell is a gastric cancer cell. In an aspect, a human cancer cell is a endometrial cancer cell.

In an aspect, a subject is a eukaryote. In an aspect, a subject is a prokaryote. In an aspect, a subject is a virus. In an aspect, a subject is an animal. In an aspect, a subject is a plant. In an aspect, a subject is a fungus. In an aspect, a subject is a mammal. In an aspect, a subject is a rodent. In an aspect, a subject is a mouse. In an aspect, a subject is a rat. In an aspect, a subject is a rabbit. In an aspect, a subject is a cat. In an aspect, a subject is a dog. In an aspect, a subject is a horse. In an aspect, a subject is a cow. In an aspect, a subject is a pig. In an aspect, a subject is a primate. In an aspect, a subject is a monkey. In an aspect, a subject is a chimpanzee. In an aspect, a subject is a human. In an aspect, a subject is a bird. In an aspect, a subject is a chicken. In an aspect, a subject is a fish. In an aspect, a subject is a reptile. In an aspect, a subject is an amphibian. In an aspect, a subject is an insect. In an aspect, a subject is an arachnid. In an aspect, a subject is a crustacean. In an aspect, a subject is a mollusk. In an aspect, a subject is a nematode. In an aspect, a subject is an annelid.

In an aspect, a subject has, or is suspected of having, cancer. In an aspect, a subject has, or is suspected of having, colorectal cancer. In an aspect, a subject has, or is suspected of having, gastric cancer. In an aspect, a subject has, or is suspected of having, endometrial cancer. In an aspect, a subject has, or is suspected of having, a genetic-based disease, disorder, or condition.

Template DNA molecules can originate from and/or be isolated from any types of cancer for use with the methods, kits, and compositions provided herein. Samples can be obtained from any type of cancer. Non-limiting examples of cancers include biliary tract cancer, bladder cancer, transitional cell carcinoma, urothelial carcinoma, brain cancer, gliomas, astrocytomas, breast carcinoma, metaplastic carcinoma, cervical cancer, cervical squamous cell carcinoma, rectal cancer, colorectal carcinoma, colon cancer, hereditary nonpolyposis colorectal cancer, colorectal adenocarcinomas, gastrointestinal stromal tumors (GISTs), endometrial carcinoma, endometrial stromal sarcomas, esophageal cancer, esophageal squamous cell carcinoma, esophageal adenocarcinoma, ocular melanoma, uveal melanoma, gallbladder carcinomas, gallbladder adenocarcinoma, renal cell carcinoma, clear cell renal cell carcinoma, transitional cell carcinoma, urothelial carcinomas, Wilms tumor, leukemia, acute lymphocytic leukemia (ALL), acute myeloid leukemia (AML), chronic lymphocytic (CLL), chronic myeloid (CIVIL), chronic myelomonocytic (CMML), liver cancer, liver carcinoma, hepatoma, hepatocellular carcinoma, cholangiocarcinoma, hepatoblastoma, Lung cancer, non-small cell lung cancer (NSCLC), mesothelioma, B-cell lymphomas, non-Hodgkin lymphoma, diffuse large B-cell lymphoma, Mantle cell lymphoma, T cell lymphomas, non-Hodgkin lymphoma, precursor T-lymphoblastic lymphoma/leukemia, peripheral T cell lymphomas, multiple myeloma, nasopharyngeal carcinoma (NPC), neuroblastoma, oropharyngeal cancer, oral cavity squamous cell carcinomas, osteosarcoma, ovarian carcinoma, pancreatic cancer, pancreatic ductal adenocarcinoma, pseudopapillary neoplasms, acinar cell carcinomas. Prostate cancer, prostate adenocarcinoma, skin cancer, melanoma, malignant melanoma, cutaneous melanoma, small intestine carcinomas, stomach cancer, gastric carcinoma, gastrointestinal stromal tumor (GIST), uterine cancer, or uterine sarcoma.

It is appreciated in the art that an “instability status” or “instability score” (e.g. repetitive nucleic acid/repetitive DNA instability status or score, microsatellite instability status or score) in the context of repetitive nucleic acids refers to a measure or determination of whether a given repetitive nucleic acid locus or population of repetitive nucleic acid loci in one or more nucleic acid samples exhibit a level or degree of mutation (e.g., variable repeat length, etc.) above, at, or below a threshold level determined for that locus or population of loci. For clarity, instability status and instability score are not interchangeable but are rather related concepts. The instability status is based on the instability score. For example, if the instability score of the sample is below or at the population trained threshold, then the sample is classified as stable sample (e.g., for MSI-MSS or MSI-Low) and if the instability score of the sample is above the population trained threshold, then the sample is classified as unstable sample (e.g., for MSI-MSI-High).

As used herein, a “threshold” refers to a separately determined value used to characterize or classify experimentally determined values.

In an aspect, a method, kit, or composition provided herein is used to assign an instability score to a sample. In an aspect, a method, kit, or composition provided herein is used to assign an instability status to a sample. In an aspect, a method, kit, or composition provided herein is used to assign an instability score to a subject. In an aspect, a method, kit, or composition provided herein is used to assign an instability status to a subject. In an aspect, an instability status is MSI-Low. In an aspect, an instability status is MSI-High.

In an aspect, the microsatellite instability score of a sample is compared to a control sample. A “control sample” refers to a sample of known composition and/or having known properties and/or known parameters (e.g., without being limiting, known tumor fraction, known sequence(s), known microsatellite instability score) that is analyzed along with or compared to a test sample in order to evaluate accuracy of an analytical procedure. In an aspect, a kit comprises a control sample.

In an aspect, a method of determining the instability status of a sample comprises the use of any composition provided herein. In an aspect, a method of determining the instability status of a sample comprises the use of any kit provided herein.

In an aspect, this disclosure provides a method of determining the instability status of a sample comprising: (a) preparing a mixture comprising: (i) a plurality of non-extensible oligonucleotides, where each of the at least two non-extensible nucleotides comprises, from 5′ to 3′, (A) a first binding sequence that is identical to the reverse complement of at least part of an upstream sequence of a DNA template molecule; (B) a second binding sequence that is identical to the reverse complement of a microsatellite repetitive sequence or a variant thereof, where the microsatellite repetitive sequence is positioned 3′ to the upstream sequence; (C) a third binding sequence that is identical to the reverse complement of at least part of a downstream sequence of the DNA template molecule, where the downstream sequence is positioned 3′ to the microsatellite repetitive sequence; and (D) a terminator sequence comprising only adenine or only thymine nucleotides, and where the terminator sequence is not identical to the reverse complement of the at least part of the downstream sequence, where the plurality of non-extensible oligonucleotides target at least two microsatellite loci; (ii) the DNA template molecule, where the DNA template molecule is obtained from the sample; (iii) a DNA polymerase; (iv) dNTPs; (v) at least two primer sets, where the at least two primer sets are capable of amplifying the at least two microsatellite loci; (b) subjecting the mixture to at least seven cycles of thermal cycling to produce at least one amplicon of each of the at least two microsatellite loci; and (c) determining the instability status of the sample based on analysis of the at least one amplicon of each of the at least two microsatellite loci obtained in step (b).

In an aspect, the MSI status of a particular sample is classified as MSI-high (MSI-H) when the microsatellite instability score for the sample is greater than 1, 2, 3, 4, 5, 6, 7, 8, 9, 10, 11, 12, 13, 14, 15, 16, 17, 18, 19, 20, 30, 40, 50, 60, 70, 80, 90, 100 unstable microsatellite loci in that sample.

In an aspect, a population trained threshold used to determine the instability status (e.g., MSI status) of a sample is 1, 2, 3, 4, 5, 6, 7, 8, 9, 10, 11, 12, 13, 14, 15, 16, 17, 18, 19, 20, 30, 40, 50, 60, 70, 80, 90, 100 or greater than 100 unstable microsatellite loci. In an aspect, the MSI status of a given sample is classified as MSI-H when the number of unstable microsatellite loci comprises at least 0.1%, at least 1%, at least 2%, at least 3%, at least 4%, at least 5%, at least 10%, at least 15%, at least 20%, or at least 25% of all microsatellite loci evaluated in that sample. In an aspect, at least 1, at least 2, at least 3, at least 4, at least 5, at least 6, at least 7, at least 8, at least 9, at least 10, at least 11, at least 12, at least 13, at least 14, at least 15, at least 20, at least 25, at least 30, at least 40, at least 50, at least 60, at least 70, at least 80, at least 90, at least 100, at least 200, at least 300, at least 400, at least 500, at least 600, at least 700, at least 800, at least 900, at least 1000, at least 1100, at least 1200, at least 1300, at least 1400, at least 1500, at least 1600, at least 1700, at least 1800, at least 1900, or at least 2000 microsatellite loci are used in determining the MSI status of a given sample.

The methods, compositions, and kits provided herein can be used to identify customized therapies to treat various types of cancer. Non-limiting examples of such cancers include biliary tract cancer, bladder cancer, transitional cell carcinoma, urothelial carcinoma, brain cancer, gliomas, astrocytomas, breast carcinoma, metaplastic carcinoma, cervical cancer, cervical squamous cell carcinoma, rectal cancer, colorectal carcinoma, colon cancer, hereditary nonpolyposis colorectal cancer, colorectal adenocarcinomas, gastrointestinal stromal tumors (GISTs), endometrial carcinoma, endometrial stromal sarcomas, esophageal cancer, esophageal squamous cell carcinoma, esophageal adenocarcinoma, ocular melanoma, uveal melanoma, gallbladder carcinomas, gallbladder adenocarcinoma, renal cell carcinoma, clear cell renal cell carcinoma, transitional cell carcinoma, urothelial carcinomas, Wilms tumor, leukemia, acute lymphocytic leukemia (ALL), acute myeloid leukemia (AML), chronic lymphocytic (CLL), chronic myeloid (CIVIL), chronic myelomonocytic (CMML), liver cancer, liver carcinoma, hepatoma, hepatocellular carcinoma, cholangiocarcinoma, hepatoblastoma, Lung cancer, non-small cell lung cancer (NSCLC), mesothelioma, B-cell lymphomas, non-Hodgkin lymphoma, diffuse large B-cell lymphoma, Mantle cell lymphoma, T cell lymphomas, non-Hodgkin lymphoma, precursor T-lymphoblastic lymphoma/leukemia, peripheral T cell lymphomas, multiple myeloma, nasopharyngeal carcinoma (NPC), neuroblastoma, oropharyngeal cancer, oral cavity squamous cell carcinomas, osteosarcoma, ovarian carcinoma, pancreatic cancer, pancreatic ductal adenocarcinoma, pseudopapillary neoplasms, acinar cell carcinomas. Prostate cancer, prostate adenocarcinoma, skin cancer, melanoma, malignant melanoma, cutaneous melanoma, small intestine carcinomas, stomach cancer, gastric carcinoma, gastrointestinal stromal tumor (GIST), uterine cancer, or uterine sarcoma.

Additionally, the methods, kits, and compositions provided herein can be used to evaluate genetic-based diseases, disorders, and/or conditions including, but not limited to, achondroplasia, alpha-1 antitrypsin deficiency, antiphospholipid syndrome, autism, autosomal dominant polycystic kidney disease, Charcot-Marie-Tooth (CMT), cri du chat, Crohn's disease, cystic fibrosis, Dercum disease, down syndrome, Duane syndrome, Duchenne muscular dystrophy, Factor V Leiden thrombophilia, familial hypercholesterolemia, familial Mediterranean fever, fragile X syndrome, Gaucher disease, hemochromatosis, hemophilia, holoprosencephaly, Huntington's disease, Klinefelter syndrome, Marfan syndrome, myotonic dystrophy, neurofibromatosis, Noonan syndrome, osteogenesis imperfecta, Parkinson's disease, phenylketonuria, Poland anomaly, porphyria, progeria, retinitis pigmentosa, severe combined immunodeficiency (scid), sickle cell disease, spinal muscular atrophy, Tay-Sachs, thalassemia, trimethylaminuria, Turner syndrome, velocardiofacial syndrome, WAGR syndrome, and Wilson disease.

In an aspect, any composition provided herein is for use in the in vitro diagnosis of a microsatellite instability score of a sample. In an aspect, any composition provided herein is for use in the in vitro diagnosis of a microsatellite instability status of a sample. In an aspect, any composition provided herein is for use in the in vitro diagnosis of a microsatellite instability score of a subject. In an aspect, any composition provided herein is for use in the in vitro diagnosis of a microsatellite instability status of a subject.

In an aspect, any kit provided herein is for use in the in vitro diagnosis of a microsatellite instability score of a sample. In an aspect, any kit provided herein is for use in the in vitro diagnosis of a microsatellite instability status of a sample. In an aspect, any kit provided herein is for use in the in vitro diagnosis of a microsatellite instability score of a subject. In an aspect, any kit provided herein is for use in the in vitro diagnosis of a microsatellite instability status of a subject.

In an aspect, a method or composition provided herein comprises a DNA template molecule. In an aspect, a method or composition provided herein comprises at least 1 DNA template molecule. In an aspect, a method or composition provided herein comprises at least 2 DNA template molecules. In an aspect, a method or composition provided herein comprises at least 5 DNA template molecules. In an aspect, a method or composition provided herein comprises at least 10 DNA template molecules. In an aspect, a method or composition provided herein comprises at least 25 DNA template molecules. In an aspect, a method or composition provided herein comprises at least 50 DNA template molecules. In an aspect, a method or composition provided herein comprises at least 100 DNA template molecules.

In an aspect, a DNA template molecule comprises a microsatellite repetitive sequence, an upstream sequence, and a downstream sequence. In an aspect, a DNA template molecule comprises, continuously from 5′ to 3′, an upstream sequence relative to a microsatellite repetitive sequence, a microsatellite repetitive sequence, and a downstream sequence relative to the microsatellite repetitive sequence.

In an aspect, a DNA template molecule comprises at least 25 nucleotides. In an aspect, a DNA template molecule comprises at least 30 nucleotides. In an aspect, a DNA template molecule comprises at least 40 nucleotides. In an aspect, a DNA template molecule comprises at least 50 nucleotides. In an aspect, a DNA template molecule comprises at least 60 nucleotides. In an aspect, a DNA template molecule comprises at least 70 nucleotides. In an aspect, a DNA template molecule comprises at least 80 nucleotides. In an aspect, a DNA template molecule comprises at least 90 nucleotides. In an aspect, a DNA template molecule comprises at least 100 nucleotides. In an aspect, a DNA template molecule comprises at least 150 nucleotides. In an aspect, a DNA template molecule comprises at least 200 nucleotides. In an aspect, a DNA template molecule comprises at least 300 nucleotides. In an aspect, a DNA template molecule comprises at least 400 nucleotides. In an aspect, a DNA template molecule comprises at least 500 nucleotides. In an aspect, a DNA template molecule comprises at least 1000 nucleotides. In an aspect, a DNA template molecule comprises at least 2500 nucleotides. In an aspect, a DNA template molecule comprises at least 5000 nucleotides.

In an aspect, a DNA template molecule comprises between 20 nucleotides and 5000 nucleotides. In an aspect, a DNA template molecule comprises between 20 nucleotides and 2500 nucleotides. In an aspect, a DNA template molecule comprises between 25 nucleotides and 2500 nucleotides. In an aspect, a DNA template molecule comprises between 25 nucleotides and 2000 nucleotides. In an aspect, a DNA template molecule comprises between 25 nucleotides and 1000 nucleotides. In an aspect, a DNA template molecule comprises between 25 nucleotides and 750 nucleotides. In an aspect, a DNA template molecule comprises between 25 nucleotides and 500 nucleotides. In an aspect, a DNA template molecule comprises between 25 nucleotides and 250 nucleotides. In an aspect, a DNA template molecule comprises between 25 nucleotides and 100 nucleotides. In an aspect, a DNA template molecule comprises between 25 nucleotides and 75 nucleotides. In an aspect, a DNA template molecule comprises between 50 nucleotides and 1000 nucleotides. In an aspect, a DNA template molecule comprises between 50 nucleotides and 500 nucleotides. In an aspect, a DNA template molecule comprises between 50 nucleotides and 250 nucleotides. In an aspect, a DNA template molecule comprises between 50 nucleotides and 100 nucleotides.

As used herein, an “upstream sequence” refers to a portion of a DNA template molecule that is immediately adjacent to the 5′ end of a microsatellite repetitive sequence. In an aspect, an upstream sequence comprises at least 20 nucleotides. In an aspect, an upstream sequence comprises at least 30 nucleotides. In an aspect, an upstream sequence comprises at least 40 nucleotides. In an aspect, an upstream sequence comprises at least 50 nucleotides. In an aspect, an upstream sequence comprises at least 60 nucleotides. In an aspect, an upstream sequence comprises at least 70 nucleotides. In an aspect, an upstream sequence comprises at least 80 nucleotides. In an aspect, an upstream sequence comprises at least 90 nucleotides. In an aspect, an upstream sequence comprises at least 100 nucleotides. In an aspect, an upstream sequence comprises between 20 nucleotides and 150 nucleotides. In an aspect, an upstream sequence comprises between 20 nucleotides and 125 nucleotides. In an aspect, an upstream sequence comprises between 20 nucleotides and 100 nucleotides. In an aspect, an upstream sequence comprises between 20 nucleotides and 75 nucleotides. In an aspect, an upstream sequence comprises between 20 nucleotides and 50 nucleotides.

As used herein, an “downstream sequence” refers to a portion of a DNA template molecule that is immediately adjacent to the 3′ end of a microsatellite repetitive sequence. In an aspect, an downstream sequence comprises at least 20 nucleotides. In an aspect, an downstream sequence comprises at least 30 nucleotides. In an aspect, an downstream sequence comprises at least 40 nucleotides. In an aspect, an downstream sequence comprises at least 50 nucleotides. In an aspect, an downstream sequence comprises at least 60 nucleotides. In an aspect, an downstream sequence comprises at least 70 nucleotides. In an aspect, an downstream sequence comprises at least 80 nucleotides. In an aspect, an downstream sequence comprises at least 90 nucleotides. In an aspect, an downstream sequence comprises at least 100 nucleotides. In an aspect, an downstream sequence comprises between 20 nucleotides and 150 nucleotides. In an aspect, an downstream sequence comprises between 20 nucleotides and 125 nucleotides. In an aspect, an downstream sequence comprises between 20 nucleotides and 100 nucleotides. In an aspect, an downstream sequence comprises between 20 nucleotides and 75 nucleotides. In an aspect, an downstream sequence comprises between 20 nucleotides and 50 nucleotides.

As used herein, a “microsatellite repetitive sequence” refers to a tract of repetitive DNA in which a certain DNA motif is repeated. As used herein, a “microsatellite locus” or “microsatellite loci” refers to a position within a template DNA molecule that comprises a microsatellite repetitive sequence.

As used herein, “unstable” or “instability,” in the context of repetitive nucleic acids, refers to a level of mutation (e.g., insertions, deletions) observed at a given repetitive nucleic acid locus or in a given population of repetitive nucleic acid loci in a sample or template DNA molecule that exceeds a threshold (e.g., a site specific threshold at a locus level; a population threshold at a sample level).

In an aspect, a DNA motif is a homopolymer repeat. As used herein, a “homopolymer motif” refers to the same nucleotide being repeated for the entire DNA motif. As a non-limiting example, the sequence 5′-AAAAAA-3′ is considered a homopolymer motif that comprises six DNA motif (A) repeats.

In an aspect, a DNA motif is a dinucleotide repeat. As used herein, a “dinucleotide repeat” refers to a block of two different nucleotides being repeated for the entire DNA motif. As a non-limiting example, the sequence 5′-ATATATAT-3′ is considered a dinucleotide repeat, with each “AT” forming one block of the dinucleotide repeat (e.g., the example repeat comprises four DNA motif (AT) repeats).

In an aspect, a DNA motif is a trinucleotide repeat. As used herein, a “trinucleotide repeat” refers to a block of three nucleotides being repeated for the entire DNA motif. As a non-limiting example, the sequence 5′-ATGATGATG-3′ is considered a trinucleotide repeat, with each “ATG” forming one block of the trinucleotide repeat (e.g., the example repeat comprises three DNA motif (ATG) repeats).

In an aspect, a DNA motif is a tetranucleotide repeat. As used herein, a “tetranucleotide repeat” refers to a block of four different nucleotides being repeated for the entire DNA motif. As a non-limiting example, the sequence 5′-ATAGATAG-3′ is considered a tetranucleotide repeat, with each “ATAG” forming one block of the tetranucleotide repeat (e.g., the example repeat comprises two DNA motif (ATAG) repeats.

In an aspect, a DNA motif is a pentanucleotide repeat. As used herein, a “pentanucleotide repeat” refers to a block of five different nucleotides being repeated for the entire DNA motif. In an aspect, a DNA motif is a hexanucleotide repeat. As used herein, a “hexanucleotide repeat” refers to a block of six different nucleotides being repeated for the entire DNA motif.

In an aspect, a microsatellite repetitive sequence comprises homopolymer repeats. In an aspect, a microsatellite repetitive sequence comprises dinucleotide repeats. In an aspect, a microsatellite repetitive sequence comprises trinucleotide repeats. In an aspect, a microsatellite repetitive sequence comprises tetranucleotide repeats. In an aspect, a microsatellite repetitive sequence comprises pentanucleotide repeats. In an aspect, a microsatellite repetitive sequence comprises hexanucleotide repeats. In an aspect, a microsatellite repetitive sequence comprises a repeat selected from the group consisting of a homopolymer repeat, a dinucleotide repeat, a trinucleotide repeat, a tetranucleotide repeat, a pentanucleotide repeat, a hexanucleotide repeat, and combinations thereof.

In an aspect, this disclosure provides variants of a “reference” microsatellite repetitive sequence. As used herein, a “variant” of a microsatellite repetitive sequence comprises the same DNA motif(s) as the reference microsatellite repetitive sequence, but a different copy number of the DNA motif(s). As a non-limiting example, for the reference microsatellite repetitive sequence 5′-AAAAAA-3′, the sequences 5′-AAAA-3′, 5′-AAAAAAAA-3′, and 5′-AAAAAAA-3′ would each be considered variants.

In an aspect, a microsatellite repetitive sequence comprises at least 4 nucleotides. In an aspect, a microsatellite repetitive sequence comprises at least 5 nucleotides. In an aspect, a microsatellite repetitive sequence comprises at least 10 nucleotides. In an aspect, a microsatellite repetitive sequence comprises at least 25 nucleotides. In an aspect, a microsatellite repetitive sequence comprises at least 30 nucleotides. In an aspect, a microsatellite repetitive sequence comprises at least 40 nucleotides. In an aspect, a microsatellite repetitive sequence comprises at least 50 nucleotides. In an aspect, a microsatellite repetitive sequence comprises at least 60 nucleotides. In an aspect, a microsatellite repetitive sequence comprises at least 70 nucleotides. In an aspect, a microsatellite repetitive sequence comprises at least 80 nucleotides. In an aspect, a microsatellite repetitive sequence comprises at least 90 nucleotides. In an aspect, a microsatellite repetitive sequence comprises at least 100 nucleotides. In an aspect, a microsatellite repetitive sequence comprises at least 125 nucleotides. In an aspect, a microsatellite repetitive sequence comprises at least 150 nucleotides. In an aspect, a microsatellite repetitive sequence comprises at least 200 nucleotides. In an aspect, a microsatellite repetitive sequence comprises between 4 nucleotides and 200 nucleotides. In an aspect, a microsatellite repetitive sequence comprises between 4 nucleotides and 150 nucleotides. In an aspect, a microsatellite repetitive sequence comprises between 4 nucleotides and 100 nucleotides. In an aspect, a microsatellite repetitive sequence comprises between 4 nucleotides and 75 nucleotides. In an aspect, a microsatellite repetitive sequence comprises between 4 nucleotides and 50 nucleotides. In an aspect, a microsatellite repetitive sequence comprises between 4 nucleotides and 25 nucleotides. In an aspect, a microsatellite repetitive sequence comprises between 4 nucleotides and 10 nucleotides. In an aspect, a microsatellite repetitive sequence comprises between 10 nucleotides and 100 nucleotides. In an aspect, a microsatellite repetitive sequence comprises between 10 nucleotides and 50 nucleotides. In an aspect, a microsatellite repetitive sequence comprises between 15 nucleotides and 40 nucleotides. In an aspect, a microsatellite repetitive sequence comprises between 15 nucleotides and 25 nucleotides.

In an aspect, the methods, kits, and compositions provided herein are suitable for use with any microsatellite locus. In an aspect, a microsatellite locus is BAT25. In an aspect, a microsatellite locus is BAT26. In an aspect, a microsatellite locus is NR21. In an aspect, a microsatellite locus is NR24. In an aspect, a microsatellite locus is Mono27. In an aspect, a microsatellite locus is NR22. In an aspect, a microsatellite locus is NR27. In an aspect, a microsatellite locus is BAT40. In an aspect, a microsatellite locus is CUL-22. In an aspect, a microsatellite locus is MET-15. In an aspect, a microsatellite locus is ATM-15. In an aspect, a microsatellite locus is RB1-13. In an aspect, a microsatellite locus is NF1-26. In an aspect, a microsatellite locus is DDR-11. In an aspect, a microsatellite locus is FANC-21. In an aspect, a microsatellite locus is MITF-14. In an aspect, a microsatellite locus is PKHD-18. In an aspect, a microsatellite locus is PTK-16. In an aspect, a microsatellite locus is RET-14. In an aspect, a microsatellite locus is CBL-17. In an aspect, a microsatellite locus is PTPN-17. In an aspect, a microsatellite locus is SMAD-18. In an aspect, a microsatellite locus is selected from the group consisting of BAT25, BAT26, NR24, NR21, Mono27, NR22, NR27, BAT40, CUL-22, MET-15, ATM-15, RB1-13, NF1-26, DDR-11, FANC-21, MITF-14, PKHD-18, PTK-16, RET-14, CBL-17, PTPN-17, and SMAD-18.

In an aspect, the methods, kits, and compositions provided herein are suitable for use with a plurality of microsatellite loci. In an aspect, the methods, kits, and compositions provided herein detect (e.g., amplify) microsatellite repeats at the BAT25, BAT26, NR21, NR24, and Mono27 loci.

As used herein, a “non-extensible oligonucleotide” or “blocker” refers to a nucleic acid molecule that prevents enzymatic extension during an amplification process such as PCR. Additional information regarding blockers can be found in U.S. Patent Application Publication No. US 2017/0067090, which is incorporated by reference herein in its entirety. In an aspect, a non-extensible oligonucleotide is a DNA molecule. In an aspect, a non-extensible oligonucleotide is an RNA molecule. In an aspect, a non-extensible oligonucleotide is a single-stranded nucleic acid molecule.

In an aspect, a non-extensible oligonucleotide comprises a binding sequence that is identical to the reverse complement of at least part of an upstream sequence. In an aspect, a non-extensible oligonucleotide comprises a binding sequence that is identical to the reverse complement of a microsatellite repetitive sequence or a variant thereof. In an aspect, a non-extensible oligonucleotide comprises a binding sequence that is identical to the reverse complement of at least part of a downstream sequence. In an aspect, a non-extensible oligonucleotide comprises a terminator sequence comprising only adenine nucleotides, where the terminator sequence is not identical to the reverse complement to at least part of a downstream sequence. In an aspect, a non-extensible oligonucleotide comprises a terminator sequence comprising only thymine nucleotides, where the terminator sequence is not identical to the reverse complement to at least part of a downstream sequence.

In an aspect, a non-extensible oligonucleotide comprises, in order from 5′ to 3′, a first binding sequence that is identical to the reverse complement of at least part of an upstream sequence, a second binding sequence that is identical to the reverse complement of a microsatellite repetitive sequence or a variant thereof, a third binding sequence that is identical to the reverse complement of at least part of a downstream sequence, and a terminator sequence comprising only adenine or only thymine nucleotides, where the terminator sequence is not identical to the reverse complement to the at least part of a downstream sequence.

In an aspect, a method, kit, or composition provided herein comprises a plurality of non-extensible oligonucleotides. In an aspect, a method, kit, or composition provided herein comprises at least 2 non-extensible oligonucleotides. In an aspect, a method, kit, or composition provided herein comprises at least 3 non-extensible oligonucleotides. In an aspect, a method, kit, or composition provided herein comprises at least 4 non-extensible oligonucleotides. In an aspect, a method, kit, or composition provided herein comprises at least 5 non-extensible oligonucleotides. In an aspect, a method, kit, or composition provided herein comprises at least 6 non-extensible oligonucleotides. In an aspect, a method, kit, or composition provided herein comprises at least 7 non-extensible oligonucleotides. In an aspect, a method, kit, or composition provided herein comprises at least 8 non-extensible oligonucleotides. In an aspect, a method, kit, or composition provided herein comprises at least 9 non-extensible oligonucleotides. In an aspect, a method, kit, or composition provided herein comprises at least 10 non-extensible oligonucleotides. In an aspect, a method, kit, or composition provided herein comprises at least 12 non-extensible oligonucleotides. In an aspect, a method, kit, or composition provided herein comprises at least 15 non-extensible oligonucleotides. In an aspect, a method, kit, or composition provided herein comprises at least 18 non-extensible oligonucleotides. In an aspect, a method, kit, or composition provided herein comprises at least 20 non-extensible oligonucleotides. In an aspect, a method, kit, or composition provided herein comprises at least 25 non-extensible oligonucleotides. In an aspect, a method, kit, or composition provided herein comprises at least 30 non-extensible oligonucleotides. In an aspect, a method, kit, or composition provided herein comprises at least 40 non-extensible oligonucleotides. In an aspect, a method, kit, or composition provided herein comprises at least 50 non-extensible oligonucleotides. In an aspect, a method, kit, or composition provided herein comprises at least 75 non-extensible oligonucleotides. In an aspect, a method, kit, or composition provided herein comprises at least 100 non-extensible oligonucleotides. In an aspect, a method, kit, or composition provided herein comprises at least 200 non-extensible oligonucleotides. In an aspect, a method, kit, or composition provided herein comprises at least 300 non-extensible oligonucleotides. In an aspect, a method, kit, or composition provided herein comprises at least 400 non-extensible oligonucleotides. In an aspect, a method, kit, or composition provided herein comprises at least 500 non-extensible oligonucleotides. In an aspect, a method, kit, or composition provided herein comprises at least 1000 non-extensible oligonucleotides.

In an aspect, a method, kit, or composition provided herein comprises between 2 and 1000 non-extensible oligonucleotides. In an aspect, a method, kit, or composition provided herein comprises between 2 and 2000 non-extensible oligonucleotides. In an aspect, a method, kit, or composition provided herein comprises between 2 and 500 non-extensible oligonucleotides. In an aspect, a method, kit, or composition provided herein comprises between 2 and 200 non-extensible oligonucleotides. In an aspect, a method, kit, or composition provided herein comprises between 2 and 100 non-extensible oligonucleotides. In an aspect, a method, kit, or composition provided herein comprises between 2 and 75 non-extensible oligonucleotides. In an aspect, a method, kit, or composition provided herein comprises between 2 and 50 non-extensible oligonucleotides. In an aspect, a method, kit, or composition provided herein comprises between 2 and 25 non-extensible oligonucleotides. In an aspect, a method, kit, or composition provided herein comprises between 5 and 50 non-extensible oligonucleotides. In an aspect, a method, kit, or composition provided herein comprises between 5 and 30 non-extensible oligonucleotides. In an aspect, a method, kit, or composition provided herein comprises between 5 and 20 non-extensible oligonucleotides. In an aspect, a method, kit, or composition provided herein comprises between 10 and 20 non-extensible oligonucleotides.

In an aspect, a method, kit, or composition provided herein comprises between (n−y) and (n+x) non-extensible oligonucleotides, where n refers to the number of DNA motif repeats in a microsatellite, and y and x are whole numbers. In an aspect, y and x are the same number. In an aspect, y and x are different numbers.

In an aspect, when multiple (e.g., at least 2, at least 3, at least 4, etc.) non-extensible oligonucleotides are provided in a method, kit, or composition, the multiple non-extensible oligonucleotides comprise identical first binding sequences and identical third binding sequences. In an aspect, when multiple non-extensible oligonucleotides are provided in a method, kit, or composition, the multiple non-extensible oligonucleotides comprise non-identical second binding sequences. In an aspect, when multiple non-extensible oligonucleotides are provided in a method, kit, or composition, the multiple non-extensible oligonucleotides comprise identical first binding sequences, identical third binding sequences, and non-identical second binding sequences.

In an aspect, a non-extensible oligonucleotide comprises at least 10 nucleotides. In an aspect, a non-extensible oligonucleotide comprises at least 12 nucleotides. In an aspect, a non-extensible oligonucleotide comprises at least 15 nucleotides. In an aspect, a non-extensible oligonucleotide comprises at least 20 nucleotides. In an aspect, a non-extensible oligonucleotide comprises at least 25 nucleotides. In an aspect, a non-extensible oligonucleotide comprises at least 30 nucleotides. In an aspect, a non-extensible oligonucleotide comprises at least 35 nucleotides. In an aspect, a non-extensible oligonucleotide comprises at least 40 nucleotides. In an aspect, a non-extensible oligonucleotide comprises at least 45 nucleotides. In an aspect, a non-extensible oligonucleotide comprises at least 50 nucleotides. In an aspect, a non-extensible oligonucleotide comprises at least 75 nucleotides. In an aspect, a non-extensible oligonucleotide comprises at least 100 nucleotides. In an aspect, a non-extensible oligonucleotide comprises between 10 nucleotides and 125 nucleotides. In an aspect, a non-extensible oligonucleotide comprises between 10 nucleotides and 100 nucleotides. In an aspect, a non-extensible oligonucleotide comprises between 10 nucleotides and 75 nucleotides. In an aspect, a non-extensible oligonucleotide comprises between 10 nucleotides and 50 nucleotides. In an aspect, a non-extensible oligonucleotide comprises between 10 nucleotides and 40 nucleotides. In an aspect, a non-extensible oligonucleotide comprises between 10 nucleotides and 30 nucleotides. In an aspect, a non-extensible oligonucleotide comprises between 12 nucleotides and 100 nucleotides.

As used herein, a “binding sequence” refers to a portion of a non-extensible oligonucleotide that is capable of hybridizing with a DNA template molecule to form a double-stranded nucleic acid molecule.

In an aspect, a binding sequence comprises at least 4 nucleotides. In an aspect, a binding sequence comprises at least 10 nucleotides. In an aspect, a binding sequence comprises at least 12 nucleotides. In an aspect, a binding sequence comprises at least 15 nucleotides. In an aspect, a binding sequence comprises at least 20 nucleotides. In an aspect, a binding sequence comprises at least 25 nucleotides. In an aspect, a binding sequence comprises at least 30 nucleotides. In an aspect, a binding sequence comprises at least 35 nucleotides. In an aspect, a binding sequence comprises at least 40 nucleotides. In an aspect, a binding sequence comprises at least 45 nucleotides. In an aspect, a binding sequence comprises at least 50 nucleotides. In an aspect, a binding sequence comprises between 4 nucleotides and 50 nucleotides. In an aspect, a binding sequence comprises between 4 nucleotides and 40 nucleotides. In an aspect, a binding sequence comprises between 4 nucleotides and 30 nucleotides. In an aspect, a binding sequence comprises between 4 nucleotides and 20 nucleotides. In an aspect, a first binding sequence comprises between 4 nucleotides and 15 nucleotides. In an aspect, a first binding sequence comprises between 4 nucleotides and 10 nucleotides. In an aspect, a first binding sequence comprises between 10 nucleotides and 50 nucleotides. In an aspect, a first binding sequence comprises between 10 nucleotides and 40 nucleotides. In an aspect, a first binding sequence comprises between 10 nucleotides and 30 nucleotides. In an aspect, a first binding sequence comprises between 10 nucleotides and 20 nucleotides.

In an aspect, a terminator sequence comprises at least 3 adenine nucleotides. In an aspect, a terminator sequence comprises at least 4 adenine nucleotides. In an aspect, a terminator sequence comprises at least 5 adenine nucleotides. In an aspect, a terminator sequence comprises at least 6 adenine nucleotides. In an aspect, a terminator sequence comprises at least 7 adenine nucleotides. In an aspect, a terminator sequence comprises at least 8 adenine nucleotides. In an aspect, a terminator sequence comprises at least 9 adenine nucleotides. In an aspect, a terminator sequence comprises at least 10 adenine nucleotides. In an aspect, a terminator sequence comprises at least 11 adenine nucleotides. In an aspect, a terminator sequence comprises at least 12 adenine nucleotides. In an aspect, a terminator sequence comprises at least 13 adenine nucleotides. In an aspect, a terminator sequence comprises at least 14 adenine nucleotides. In an aspect, a terminator sequence comprises at least 15 adenine nucleotides. In an aspect, a terminator sequence comprises at least 20 adenine nucleotides. In an aspect, a terminator sequence comprises between 3 adenine nucleotides and 25 adenine nucleotides. In an aspect, a terminator sequence comprises between 3 adenine nucleotides and 20 adenine nucleotides. In an aspect, a terminator sequence comprises between 3 adenine nucleotides and 15 adenine nucleotides. In an aspect, a terminator sequence comprises between 3 adenine nucleotides and 10 adenine nucleotides. In an aspect, a terminator sequence comprises between 3 adenine nucleotides and 8 adenine nucleotides.

In an aspect, a terminator sequence comprises at least 3 thymine nucleotides. In an aspect, a terminator sequence comprises at least 4 thymine nucleotides. In an aspect, a terminator sequence comprises at least 5 thymine nucleotides. In an aspect, a terminator sequence comprises at least 6 thymine nucleotides. In an aspect, a terminator sequence comprises at least 7 thymine nucleotides. In an aspect, a terminator sequence comprises at least 8 thymine nucleotides. In an aspect, a terminator sequence comprises at least 9 thymine nucleotides. In an aspect, a terminator sequence comprises at least 10 thymine nucleotides. In an aspect, a terminator sequence comprises at least 11 thymine nucleotides. In an aspect, a terminator sequence comprises at least 12 thymine nucleotides. In an aspect, a terminator sequence comprises at least 13 thymine nucleotides. In an aspect, a terminator sequence comprises at least 14 thymine nucleotides. In an aspect, a terminator sequence comprises at least 15 thymine nucleotides. In an aspect, a terminator sequence comprises at least 20 thymine nucleotides. In an aspect, a terminator sequence comprises between 3 thymine nucleotides and 25 thymine nucleotides. In an aspect, a terminator sequence comprises between 3 thymine nucleotides and 20 thymine nucleotides. In an aspect, a terminator sequence comprises between 3 thymine nucleotides and 15 thymine nucleotides. In an aspect, a terminator sequence comprises between 3 thymine nucleotides and 10 thymine nucleotides. In an aspect, a terminator sequence comprises between 3 thymine nucleotides and 8 thymine nucleotides.

In an aspect, a terminator sequence is not identical to the reverse complement of a downstream sequence.

In an aspect, a composition, method, or kit provided herein comprises a DNA polymerase. In an aspect, a DNA polymerase is selected form the group consisting of phi29 DNA polymerase, DNA polymerase 1, large (Klenow) fragment, Klenow fragment, Bst DNA polymerase, T4 DNA polymerase, T7 DNA polymerase, Taq polymerase, Phusion® polymerase, Q5® polymerase, KAPA HiFi polymerase, Vent® DNA polymerase, LongAmp® Taq DNA polymerase, and OneTaq® DNA polymerase.

In an aspect, a composition, method, or kit comprises one or more reagents necessary for DNA polymerase activity. Non-limiting examples of reagents necessary for DNA polymerase activity include dNTPs, buffers, magnesium, phosphatase, betaine, dimethyl sulfoxide, and tetramethylammonium chloride.

In an aspect, a method, kit, or composition comprises at least one primer. As used herein, the term “primer” refers to a single-stranded nucleic acid molecule used for initiating DNA synthesis with a DNA polymerase enzyme. In an aspect, a primer is a forward primer. A forward primer binds to the (−) strand of a DNA molecule. In an aspect, a primer is a reverse primer. A reverse primer binds to the (+) strand of a DNA molecule. In an aspect, a non-extensible oligonucleotide hybridizes to a DNA template molecule between a forward primer and a reverse primer. When a forward primer and a reverse primer are designed to amplify a target locus together, they are collectively termed a “primer set.”

In an aspect, a method, kit, or composition provided herein comprises at least one forward primer. In an aspect, a method, kit, or composition provided herein comprises at least one reverse primer.

In an aspect, the sequence at the 3′ end of a forward primer and the sequence at the 5′ end of the first binding sequence of a non-extensible oligonucleotide are identical. This identical sequence is termed an “overlap” or “overlapping sequence” and the forward primer and non-extensible oligonucleotide can compete to bind the DNA template molecule. When a primer and a non-extensible oligonucleotide comprise an overlapping subsequence, the primer also has a “non-overlapping subsequence,” which refers to the portion of the primer sequence that does not overlap with the non-extensible oligonucleotide sequence.

In an aspect, a forward primer sequence and a first binding sequence of a non-extensible oligonucleotide comprise an overlap of between 2 nucleotides and 15 nucleotides. In an aspect, a forward primer sequence and a first binding sequence of a non-extensible oligonucleotide comprise an overlap of between 2 nucleotides and 12 nucleotides. In an aspect, a forward primer sequence and a first binding sequence of a non-extensible oligonucleotide comprise an overlap of between 2 nucleotides and 10 nucleotides. In an aspect, a forward primer sequence and a first binding sequence of a non-extensible oligonucleotide comprise an overlap of between 2 nucleotides and 7 nucleotides. In an aspect, a forward primer sequence and a first binding sequence of a non-extensible oligonucleotide comprise an overlap of between 5 nucleotides and 15 nucleotides. In an aspect, a forward primer sequence and a first binding sequence of a non-extensible oligonucleotide comprise an overlap of between 5 nucleotides and 10 nucleotides. In an aspect, a forward primer sequence and a first binding sequence of a non-extensible oligonucleotide comprise an overlap of at least 2 nucleotides. In an aspect, a forward primer sequence and a first binding sequence of a non-extensible oligonucleotide comprise an overlap of at least 3 nucleotides. In an aspect, a forward primer sequence and a first binding sequence of a non-extensible oligonucleotide comprise an overlap of at least 4 nucleotides. In an aspect, a forward primer sequence and a first binding sequence of a non-extensible oligonucleotide comprise an overlap of at least 5 nucleotides. In an aspect, a forward primer sequence and a first binding sequence of a non-extensible oligonucleotide comprise an overlap of at least 6 nucleotides. In an aspect, a forward primer sequence and a first binding sequence of a non-extensible oligonucleotide comprise an overlap of at least 7 nucleotides. In an aspect, a forward primer sequence and a first binding sequence of a non-extensible oligonucleotide comprise an overlap of at least 8 nucleotides. In an aspect, a forward primer sequence and a first binding sequence of a non-extensible oligonucleotide comprise an overlap of at least 9 nucleotides. In an aspect, a forward primer sequence and a first binding sequence of a non-extensible oligonucleotide comprise an overlap of at least 10 nucleotides.

In an aspect, a non-extensible oligonucleotide sequence and a reverse primer sequence do not overlap.

In an aspect, a primer is a DNA molecule. In an aspect, a primer is an RNA molecule. In an aspect, a primer comprises at least 5 nucleotides. In an aspect, a primer comprises at least 10 nucleotides. In an aspect, a primer comprises at least 15 nucleotides. In an aspect, a primer comprises at least 20 nucleotides. In an aspect, a primer comprises at least 25 nucleotides. In an aspect, a primer comprises at least 30 nucleotides. In an aspect, a primer comprises at least 35 nucleotides. In an aspect, a primer comprises at least 40 nucleotides. In an aspect, a primer comprises at least 50 nucleotides. In an aspect, a primer comprises at least 70 nucleotides. In an aspect, a primer comprises between 5 nucleotides and 100 nucleotides. In an aspect, a primer comprises between 5 nucleotides and 90 nucleotides. In an aspect, a primer comprises between 5 nucleotides and 80 nucleotides. In an aspect, a primer comprises between 5 nucleotides and 70 nucleotides. In an aspect, a primer comprises between 5 nucleotides and 60 nucleotides. In an aspect, a primer comprises between 5 nucleotides and 50 nucleotides. In an aspect, a primer comprises between 5 nucleotides and 40 nucleotides. In an aspect, a primer comprises between 5 nucleotides and 30 nucleotides. In an aspect, a primer comprises between 5 nucleotides and 25 nucleotides. In an aspect, a primer comprises between 5 nucleotides and 20 nucleotides. In an aspect, a primer comprises between 10 nucleotides and 70 nucleotides. In an aspect, a primer comprises between 10 nucleotides and 50 nucleotides. In an aspect, a primer comprises between 10 nucleotides and 30 nucleotides.

In an aspect, a primer further comprises an adapter. As used herein, an “adapter” refers to a known nucleic acid sequence that can serve a variety of purposes. In an aspect, an adapter comprises a flow cell binding sequences for high-throughput sequencing. In an aspect, an adapter comprises a sequencing primer site. In an aspect, an adapter comprises a sample index to tag and identify a given library for high-throughput sequencing. In an aspect, an adapter comprises a molecular barcode to uniquely tag an individual molecule within a library.

In an aspect, a forward primer comprises a forward adapter at its 5′ end. In an aspect, a reverse primer comprises a reverse adapter at its 5′ end. In an aspect, a forward adapter, a reverse adapter, or both, comprises a sequence selected from the group consisting of a flow cell binding sequence, a sequencing primer sit, a sample index, a molecular barcode, or any combination thereof.

In an aspect, a method provided herein comprises ligating at least one adapter to an amplicon. As used herein, an “amplicon” refers to a nucleic acid molecule that has been amplified from a DNA template molecule, for example, during PCR. In an aspect, an amplicon comprises only a fraction of a DNA template molecule (e.g., the amplicon is shorter in length than the DNA template molecule). In an aspect, an amplicon comprises at least 25 nucleotides. In an aspect, an amplicon comprises at least 50 nucleotides. In an aspect, an amplicon comprises at least 75 nucleotides. In an aspect, an amplicon comprises at least 100 nucleotides. In an aspect, an amplicon comprises at least 200 nucleotides. In an aspect, an amplicon comprises at least 500 nucleotides.

In one aspect, this disclosure provides a method for selectively inhibiting a polymerase chain reaction amplification of at least one DNA template molecule comprising at least one microsatellite repetitive sequence, the method comprising: (a) preparing a mixture comprising: (i) a plurality of non-extensible oligonucleotides, where each of the at least two non-extensible nucleotides comprises, from 5′ to 3′, (A) a first binding sequence that is identical to the reverse complement of at least part of an upstream sequence of a DNA template molecule; (B) a second binding sequence that is identical to the reverse complement of a microsatellite repetitive sequence or a variant thereof, where the microsatellite repetitive sequence is positioned 3′ to the upstream sequence; (C) a third binding sequence that is identical to the reverse complement of at least part of a downstream sequence of the DNA template molecule, where the downstream sequence is positioned 3′ to the microsatellite repetitive sequence; and (D) a terminator sequence comprising only adenine or only thymine nucleotides, and where the terminator sequence is not identical to the reverse complement of the at least part of the downstream sequence; (ii) the at least one DNA template molecule; (iii) a DNA polymerase; (iv) dNTPs; (v) a forward primer and a reverse primer, where the forward and reverse primer are capable of amplifying the at least one DNA template molecule; and (b) subjecting the mixture to at least seven cycles of thermal cycling to produce at least one amplicon of at least one member of the group of DNA template molecules.

In an aspect, a method further comprises (c) subjecting at least one amplicon to high-throughput sequencing. In an aspect, a method further comprises (c) ligating an adapter sequence to the at least one amplicon; and (d) subjecting the amplicon from step (c) to high-throughput sequencing.

As used herein, “high-throughput sequencing” refers to any sequencing method that is capable of sequencing multiple (e.g., tens, hundreds, thousands, millions, hundreds of millions) DNA molecules in parallel. In an aspect, Sanger sequencing is not high-throughput sequencing. In an aspect, high-throughput sequencing comprises the use of a sequencing-by-synthesis (SBS) flow cell. In an aspect, an SBS flow cell is selected from the group consisting of an Illumina SBS flow cell and a Pacific Biosciences (PacBio) SBS flow cell. In an aspect, high-throughput sequencing is performed via electrical current measurements in conjunction with an Oxford nanopore.

In an aspect, a mixture further comprises an intercalating dye. In an aspect, an intercalating dye is selected from the group consisting of ethidium bromide, propidium iodide, and SYBR Green.

In an aspect, a mixture further comprises at least one TaqMan® probe. In an aspect, a mixture further comprises at least two TaqMan® probes. In an aspect, a mixture further comprises at least five TaqMan® probes. In an aspect, a mixture further comprises at least ten TaqMan® probes.

As used herein, “thermal cycling” refers to a controlled set of timed temperature changes. One “cycle” of thermal cycling comprises at least two stages. The first stage of a cycle comprises a first temperature maintained for a desired amount of time, and the second stage of a cycle comprises a second temperature maintained for a desired amount of time. In an aspect, a cycle further comprises a third stage comprising a third temperature maintained for a desired amount of time. In an aspect, a cycle further comprises a fourth stage comprising a fourth temperature maintained for a desired amount of time. Often, thermal cycling comprises repeating the same cycle several times.

In an aspect, a first, second, third, or fourth stage of a cycle comprises a temperature of less than 60° C. In an aspect, a first, second, third, or fourth stage of a cycle comprises a temperature of less than 70° C. In an aspect, a first, second, third, or fourth stage of a cycle comprises a temperature of less than 75° C. In an aspect, a first, second, third, or fourth stage of a cycle comprises a temperature of less than 80° C. In an aspect, a first, second, third, or fourth stage of a cycle comprises a temperature of less than 90° C. In an aspect, a first, second, third, or fourth stage of a cycle comprises a temperature of greater than 60° C. In an aspect, a first, second, third, or fourth stage of a cycle comprises a temperature of greater than 70° C. In an aspect, a first, second, third, or fourth stage of a cycle comprises a temperature of greater than 75° C. In an aspect, a first, second, third, or fourth stage of a cycle comprises a temperature of greater than 80° C. In an aspect, a first, second, third, or fourth stage of a cycle comprises a temperature of greater than 90° C.

In an aspect, a first, second, third, or fourth stage of a cycle lasts for at least 1 second. In an aspect, a first, second, third, or fourth stage of a cycle lasts for at least 10 seconds. In an aspect, a first, second, third, or fourth stage of a cycle lasts for at least 30 seconds. In an aspect, a first, second, third, or fourth stage of a cycle lasts for at least 1 minute. In an aspect, a first, second, third, or fourth stage of a cycle lasts for at least 2 minutes. In an aspect, a first, second, third, or fourth stage of a cycle lasts for at least 10 minutes. In an aspect, a first, second, third, or fourth stage of a cycle lasts for at least 15 minutes. In an aspect, a first, second, third, or fourth stage of a cycle lasts for at least 30 minutes. In an aspect, a first, second, third, or fourth stage of a cycle lasts for at least 1 hour. In an aspect, a first, second, third, or fourth stage of a cycle lasts for at least 2 hours.

In an aspect, a first, second, third, or fourth stage of a cycle lasts for between 1 second and 3 hours. In an aspect, a first, second, third, or fourth stage of a cycle lasts for between 1 second and 2 hours. In an aspect, a first, second, third, or fourth stage of a cycle lasts for between 1 second and 1 hour. In an aspect, a first, second, third, or fourth stage of a cycle lasts for between 1 second and 30 minutes. In an aspect, a first, second, third, or fourth stage of a cycle lasts for between 1 second and 20 minutes. In an aspect, a first, second, third, or fourth stage of a cycle lasts for between 1 second and 15 minutes. In an aspect, a first, second, third, or fourth stage of a cycle lasts for between 1 second and 10 minutes. In an aspect, a first, second, third, or fourth stage of a cycle lasts for between 1 second and 5 minutes. In an aspect, a first, second, third, or fourth stage of a cycle lasts for between 1 second and 2 minutes. In an aspect, a first, second, third, or fourth stage of a cycle lasts for between 1 second and 1 minute. In an aspect, a first, second, third, or fourth stage of a cycle lasts for between 1 second and 30 seconds.

In an aspect, thermal cycling comprises at least 1 cycle. In an aspect, thermal cycling comprises at least 2 cycles. In an aspect, thermal cycling comprises at least 3 cycles. In an aspect, thermal cycling comprises at least 4 cycles. In an aspect, thermal cycling comprises at least 5 cycles. In an aspect, thermal cycling comprises at least 6 cycles. In an aspect, thermal cycling comprises at least 7 cycles. In an aspect, thermal cycling comprises at least 8 cycles. In an aspect, thermal cycling comprises at least 9 cycles. In an aspect, thermal cycling comprises at least 10 cycles. In an aspect, thermal cycling comprises at least 15 cycles. In an aspect, thermal cycling comprises at least 20 cycles. In an aspect, thermal cycling comprises at least 25 cycles. In an aspect, thermal cycling comprises at least 30 cycles. In an aspect, thermal cycling comprises at least 40 cycles. In an aspect, thermal cycling comprises at least 50 cycles.

In an aspect, thermal cycling comprises between 1 cycle and 60 cycles. In an aspect, thermal cycling comprises between 1 cycle and 50 cycles. In an aspect, thermal cycling comprises between 1 cycle and 40 cycles. In an aspect, thermal cycling comprises between 1 cycle and 30 cycles. In an aspect, thermal cycling comprises between 1 cycle and 20 cycles. In an aspect, thermal cycling comprises between 1 cycle and 10 cycles. In an aspect, thermal cycling comprises between 1 cycle and 5 cycles. In an aspect, thermal cycling comprises between 2 cycles and 60 cycles. In an aspect, thermal cycling comprises between 2 cycles and 40 cycles. In an aspect, thermal cycling comprises between 2 cycles and 20 cycles. In an aspect, thermal cycling comprises between 2 cycles and 10 cycles. In an aspect, thermal cycling comprises between 2 cycles and 8 cycles. In an aspect, thermal cycling comprises between 20 cycles and 60 cycles. In an aspect, thermal cycling comprises between 20 cycles and 40 cycles.

In an aspect, each cycle of thermal cycling comprises (a) a first stage comprising a temperature of at least 75° C. for between one second and one hour; and (b) a second stage comprising a temperature of less than 75° C. for between one second and two hours. In an aspect, each cycle of thermal cycling comprises (a) a first stage comprising a temperature of at least 80° C. for between one second and one hour; and (b) a second stage comprising a temperature of less than 80° C. for between one second and two hours. In an aspect, each cycle of thermal cycling comprises (a) a first stage comprising a temperature of at least 90° C. for between one second and one hour; and (b) a second stage comprising a temperature of less than 90° C. for between one second and two hours.

As used herein, “analytical sensitivity” refers to the ability to detect a nucleic acid sequence or molecule of interest when the nucleic acid sequence or molecule makes up a given percentage of the total nucleic acid sequence or molecules in a mixture or solution. As a non-limiting an example, if a method can detect a nucleic acid molecule when it makes up 10% of the total nucleic acid molecules in a mixture, the method would have an analytical sensitivity of 10%. As another non-limiting an example, if a method can detect a nucleic acid molecule when it makes up 3% of the total nucleic acid molecules in a mixture, the method would have an analytical sensitivity of 3%.

In an aspect, a method or kit provided herein has an analytical sensitivity of less than or equal to 5%. In an aspect, a method or kit provided herein has an analytical sensitivity of less than or equal to 4%. In an aspect, a method or kit provided herein has an analytical sensitivity of less than or equal to 3%. In an aspect, a method or kit provided herein has an analytical sensitivity of less than or equal to 2%. In an aspect, a method or kit provided herein has an analytical sensitivity of less than or equal to 1%. In an aspect, a method or kit provided herein has an analytical sensitivity of less than or equal to 0.75%. In an aspect, a method or kit provided herein has an analytical sensitivity of less than or equal to 0.5%. In an aspect, a method or kit provided herein has an analytical sensitivity of less than or equal to 0.25%. In an aspect, a method or kit provided herein has an analytical sensitivity of less than or equal to 0.225%. In an aspect, a method or kit provided herein has an analytical sensitivity of less than or equal to 0.2%. In an aspect, a method or kit provided herein has an analytical sensitivity of less than or equal to 0.175%. In an aspect, a method or kit provided herein has an analytical sensitivity of less than or equal to 0.15%. In an aspect, a method or kit provided herein has an analytical sensitivity of less than or equal to 0.125%. In an aspect, a method or kit provided herein has an analytical sensitivity of less than or equal to 0.1%.

In one aspect, this disclosure provides a kit comprising: (a) a plurality of non-extensible oligonucleotides, where each of the plurality of non-extensible nucleotides comprises, from 5′ to 3′, (i) a first binding sequence that is identical to the reverse complement of at least part of an upstream sequence of a DNA template molecule; (ii) a second binding sequence that is identical to the reverse complement of a microsatellite repetitive sequence or a variant thereof, where the microsatellite repetitive sequence is positioned 3′ to the upstream sequence; (iii) a third binding sequence that is identical to the reverse complement of at least part of a downstream sequence of the DNA template molecule, where the downstream sequence is positioned 3′ to the microsatellite repetitive sequence; and (iv) a terminator sequence comprising only adenine or only thymine nucleotides, and where the terminator sequence is not identical to the reverse complement of the at least part of the downstream sequence; and (b) a forward primer and a reverse primer, where the forward and reverse primer are capable of amplifying the DNA template molecule.

In an aspect, a plurality of non-extensible oligonucleotides comprises 3 or more non-extensible oligonucleotides. In an aspect, a plurality of non-extensible oligonucleotides comprises 4 or more non-extensible oligonucleotides. In an aspect, a plurality of non-extensible oligonucleotides comprises 5 or more non-extensible oligonucleotides. In an aspect, a plurality of non-extensible oligonucleotides comprises 6 or more non-extensible oligonucleotides. In an aspect, a plurality of non-extensible oligonucleotides comprises 7 or more non-extensible oligonucleotides. In an aspect, a plurality of non-extensible oligonucleotides comprises 8 or more non-extensible oligonucleotides. In an aspect, a plurality of non-extensible oligonucleotides comprises 9 or more non-extensible oligonucleotides. In an aspect, a plurality of non-extensible oligonucleotides comprises 10 or more non-extensible oligonucleotides. In an aspect, a plurality of non-extensible oligonucleotides comprises 11 or more non-extensible oligonucleotides. In an aspect, a plurality of non-extensible oligonucleotides comprises 12 or more non-extensible oligonucleotides. In an aspect, a plurality of non-extensible oligonucleotides comprises 13 or more non-extensible oligonucleotides. In an aspect, a plurality of non-extensible oligonucleotides comprises 14 or more non-extensible oligonucleotides. In an aspect, a plurality of non-extensible oligonucleotides comprises 15 or more non-extensible oligonucleotides. In an aspect, a plurality of non-extensible oligonucleotides comprises 20 or more non-extensible oligonucleotides. In an aspect, a plurality of non-extensible oligonucleotides comprises 25 or more non-extensible oligonucleotides. In an aspect, a plurality of non-extensible oligonucleotides comprises 30 or more non-extensible oligonucleotides. In an aspect, a plurality of non-extensible oligonucleotides comprises 35 or more non-extensible oligonucleotides. In an aspect, a plurality of non-extensible oligonucleotides comprises 40 or more non-extensible oligonucleotides. In an aspect, a plurality of non-extensible oligonucleotides comprises 45 or more non-extensible oligonucleotides. In an aspect, a plurality of non-extensible oligonucleotides comprises 50 or more non-extensible oligonucleotides. In an aspect, a plurality of non-extensible oligonucleotides comprises 60 or more non-extensible oligonucleotides. In an aspect, a plurality of non-extensible oligonucleotides comprises 70 or more non-extensible oligonucleotides. In an aspect, a plurality of non-extensible oligonucleotides comprises 80 or more non-extensible oligonucleotides. In an aspect, a plurality of non-extensible oligonucleotides comprises 90 or more non-extensible oligonucleotides. In an aspect, a plurality of non-extensible oligonucleotides comprises 100 or more non-extensible oligonucleotides.

In an aspect, a kit or composition comprises a plurality of forward primers and a plurality of reverse primers. In an aspect, a kit or composition comprises 2 or more forward primers and 2 or more reverse primers. In an aspect, a kit or composition comprises 3 or more forward primers and 3 or more reverse primers. In an aspect, a kit or composition comprises 4 or more forward primers and 4 or more reverse primers. In an aspect, a kit or composition comprises 5 or more forward primers and 5 or more reverse primers. In an aspect, a kit or composition comprises 6 or more forward primers and 6 or more reverse primers. In an aspect, a kit or composition comprises 7 or more forward primers and 7 or more reverse primers. In an aspect, a kit or composition comprises 8 or more forward primers and 8 or more reverse primers. In an aspect, a kit or composition comprises 9 or more forward primers and 9 or more reverse primers. In an aspect, a kit or composition comprises 10 or more forward primers and 10 or more reverse primers. In an aspect, a kit or composition comprises 11 or more forward primers and 11 or more reverse primers. In an aspect, a kit or composition comprises 12 or more forward primers and 12 or more reverse primers. In an aspect, a kit or composition comprises 13 or more forward primers and 13 or more reverse primers. In an aspect, a kit or composition comprises 14 or more forward primers and 14 or more reverse primers. In an aspect, a kit or composition comprises 15 or more forward primers and 15 or more reverse primers. In an aspect, a kit or composition comprises 20 or more forward primers and 20 or more reverse primers. In an aspect, a kit or composition comprises 25 or more forward primers and 25 or more reverse primers. In an aspect, a kit or composition comprises 30 or more forward primers and 30 or more reverse primers. In an aspect, a kit or composition comprises 35 or more forward primers and 35 or more reverse primers. In an aspect, a kit or composition comprises 40 or more forward primers and 40 or more reverse primers. In an aspect, a kit or composition comprises 45 or more forward primers and 45 or more reverse primers. In an aspect, a kit or composition comprises 50 or more forward primers and 50 or more reverse primers. In an aspect, a kit or composition comprises 60 or more forward primers and 60 or more reverse primers. In an aspect, a kit or composition comprises 70 or more forward primers and 70 or more reverse primers. In an aspect, a kit or composition comprises 80 or more forward primers and 80 or more reverse primers. In an aspect, a kit or composition comprises 90 or more forward primers and 90 or more reverse primers. In an aspect, a kit or composition comprises 100 or more forward primers and 100 or more reverse primers.

In an aspect, a kit comprises a DNA polymerase. In an aspect, a kit comprises water. In an aspect, a kit comprises dNTPs (deoxyribonucleotide triphosphates).

In an aspect, a non-extensible oligonucleotide is provided in a kit in lyophilized form. In an aspect, a forward primer is provided in a kit in lyophilized form. In an aspect, a reverse primer is provided in a kit in lyophilized form.

In an aspect, a method or kit provided herein comprises an analytical sensitivity for a microsatellite locus of less than or equal to 0.25%. In an aspect, a method or kit provided herein comprises an analytical sensitivity for a microsatellite locus of less than or equal to 0.2%. In an aspect, a method or kit provided herein comprises an analytical sensitivity for a microsatellite locus of less than or equal to 0.175%. In an aspect, a method or kit provided herein comprises an analytical sensitivity for a microsatellite locus of less than or equal to 0.15%. In an aspect, a method or kit provided herein comprises an analytical sensitivity for a microsatellite locus of less than or equal to 0.125%. In an aspect, a method or kit provided herein comprises an analytical sensitivity for a microsatellite locus of less than or equal to 0.1%.

The following exemplary, non-limiting embodiments are envisioned:

1. A composition comprising:
(a) a DNA template molecule comprising, continuously from 5′ to 3′
   (i) an upstream sequence upstream to a microsatellite repetitive sequence;
   (ii) the microsatellite repetitive sequence; and
   (iii) a downstream sequence downstream to the microsatellite repetitive sequence; and
(b) a plurality of non-extensible oligonucleotides, wherein each of the plurality of non-extensible oligonucleotides comprises, from 5′ to 3′,
   (i) a first binding sequence that is identical to the reverse complement of at least part of the upstream sequence;
   (ii) a second binding sequence that is identical to the reverse complement of the microsatellite repetitive sequence or a variant thereof;
   (iii) a third binding sequence that is identical to the reverse complement of at least part of the downstream sequence; and
   (iv) a terminator sequence comprising only adenine or only thymine nucleotides, and wherein the terminator sequence is not identical to the reverse complement of the at least part of the downstream sequence.
      2. A composition comprising a plurality of non-extensible oligonucleotides, wherein each of the plurality of non-extensible nucleotides comprises, from 5′ to 3′,
   (i) a first binding sequence that is identical to the reverse complement of at least part of an upstream sequence of a DNA template molecule;
   (ii) a second binding sequence that is identical to the reverse complement of a microsatellite repetitive sequence or a variant thereof, wherein the microsatellite repetitive sequence is positioned 3′ to the upstream sequence;
   (iii) a third binding sequence that is identical to the reverse complement of at least part of a downstream sequence of the DNA template molecule, wherein the downstream sequence is positioned 3′ to the microsatellite repetitive sequence; and
   (iv) a terminator sequence comprising only adenine or only thymine nucleotides, and wherein the terminator sequence is not identical to the reverse complement of the at least part of the downstream sequence.
      3. The composition of embodiment 2, wherein the composition further comprises the DNA template molecule.
      4. The composition of any one of embodiments 1-3, wherein the composition further comprises a DNA polymerase.
      5. The composition of embodiment 4, wherein the at least one DNA polymerase is selected from the group consisting of phi29 DNA polymerase, DNA polymerase 1, large (Klenow) fragment, Klenow fragment, Bst DNA polymerase, T4 DNA polymerase, T7 DNA polymerase, Taq polymerase, Phusion® polymerase, Q5® polymerase, KAPA HiFi polymerase, Vent® DNA polymerase, LongAmp® Taq DNA polymerase, and OneTaq® DNA polymerase.
      6. The composition of embodiment 4 or 5, wherein the composition further comprises one or more reagents necessary for DNA polymerase activity.
      7. The composition of any one of embodiments 1-6, wherein the plurality of non-extensible oligonucleotides comprises non-identical second binding sequences.
      8. The composition of any one of embodiments 1-7, wherein the plurality of non-extensible oligonucleotides comprises identical first binding sequences and identical third binding sequences.
      9. The composition of any one of embodiments 1-8, wherein the microsatellite repetitive sequence comprises homopolymer repeats.
      10. The composition of any one of embodiments 1-8, wherein the microsatellite repetitive sequence comprises dinucleotide repeats.
      11. The composition of any one of embodiments 1-8, wherein the microsatellite repetitive sequence comprises trinucleotide repeats.
      12. The composition of any one of embodiments 1-8, wherein the microsatellite repetitive sequence comprises tetranucleotide repeats.
      13. The composition of any one of embodiments 1-12, wherein the terminator sequence comprises between 4 nucleotides and 100 nucleotides.
      14. The composition of any one of embodiments 1-13, wherein the composition further comprises a forward primer and a reverse primer.
      15. The composition of embodiment 14, wherein the forward primer and the plurality of non-extensible oligonucleotides comprise an overlapping sequence.
      16. The composition of embodiment 15, wherein the overlapping sequence comprises between 2 nucleotides and 15 nucleotides.
      17. The composition of any one of embodiments 1-16, wherein the plurality of non-extensible oligonucleotides comprises between 2 and 100 non-extensible oligonucleotides.
      18. A method for selectively inhibiting a polymerase chain reaction amplification of at least one DNA template molecule comprising at least one microsatellite repetitive sequence, the method comprising:
(a) preparing a mixture comprising:
   (i) a plurality of non-extensible oligonucleotides, wherein each of the plurality of non-extensible oligonucleotides comprises, from 5′ to 3′,
      (A) a first binding sequence that is identical to the reverse complement of at least part of an upstream sequence of a DNA template molecule;
      (B) a second binding sequence that is identical to the reverse complement of a microsatellite repetitive sequence or a variant thereof, wherein the microsatellite repetitive sequence is positioned 3′ to the upstream sequence;
      (C) a third binding sequence that is identical to the reverse complement of at least part of a downstream sequence of the DNA template molecule, wherein the downstream sequence is positioned 3′ to the microsatellite repetitive sequence; and
      (D) a terminator sequence comprising only adenine or only thymine nucleotides, and wherein the terminator sequence is not identical to the reverse complement of the at least part of the downstream sequence;
   (ii) the at least one DNA template molecule;
   (iii) a DNA polymerase;
   (iv) dNTPs;
   (v) a forward primer and a reverse primer, wherein the forward and reverse primer are capable of amplifying at least one member of the at least one DNA template molecule; and
(b) subjecting the mixture to at least seven cycles of thermal cycling to produce at least one amplicon of the at least one DNA template molecule.
   19. The method of embodiment 18, wherein the mixture further comprises an intercalating dye.
   20. The method of embodiment 18 or 19, wherein the mixture further comprises at least one TaqMan® probe.
   21. The method of any one of embodiments 18-20, wherein the forward primer comprises a forward adapter at its 5′ end.
   22. The method of any one of embodiments 18-21, wherein the reverse primer comprises a reverse adapter at its 5′ end.
   23. The method of embodiment 21 or 22, wherein the method further comprises: (c) subjecting the at least one amplicon to high-throughput sequencing.
   24. The method of any one of embodiments 18-20, wherein the method further comprises: (c) ligating an adapter sequence to the at least one amplicon; and (d) subjecting the amplicon from step (c) to high-throughput sequencing.
   25. The method of any one of embodiments 18-24, wherein the at least one DNA template molecule comprises at least 10 DNA template molecules.
   26. The method of any one of embodiments 18-25, wherein the DNA polymerase is selected from the group consisting of phi29 DNA polymerase, DNA polymerase 1, large (Klenow) fragment, Klenow fragment, Bst DNA polymerase, T4 DNA polymerase, T7 DNA polymerase, Taq polymerase, Phusion® polymerase, Q5® polymerase, KAPA HiFi polymerase, Vent® DNA polymerase, LongAmp® Taq DNA polymerase, and OneTaq® DNA polymerase.
   27. The method of any one of embodiments 18-26, wherein the at least one amplicon comprises at least 50 nucleotides.
   28. A kit comprising:
(a) a plurality of non-extensible oligonucleotides, wherein each of the plurality of non-extensible nucleotides comprises, from 5′ to 3′,
   (i) a first binding sequence that is identical to the reverse complement of at least part of an upstream sequence of a DNA template molecule;
   (ii) a second binding sequence that is identical to the reverse complement of a microsatellite repetitive sequence or a variant thereof, wherein the microsatellite repetitive sequence is positioned 3′ to the upstream sequence;
   (iii) a third binding sequence that is identical to the reverse complement of at least part of a downstream sequence of the DNA template molecule, wherein the downstream sequence is positioned 3′ to the microsatellite repetitive sequence; and
   (iv) a terminator sequence comprising only adenine or only thymine nucleotides, and wherein the terminator sequence is not identical to the reverse complement of the at least part of the downstream sequence; and
(b) a forward primer and a reverse primer, wherein the forward and reverse primer are capable of amplifying the DNA template molecule.
   29. The kit of embodiment 28, wherein the kit comprises a plurality of forward primers and a plurality of reverse primers.
   30. The kit of embodiment 28 or 29, wherein the kit comprises 5 or more forward primers and 5 or more reverse primers.
   31. The kit of any one of embodiments 28-30, wherein the kit comprises 10 or more non-extensible oligonucleotides.

32. The kit of any one of embodiments 28-31, wherein the DNA template molecule is a human DNA template molecule.

33. A method of determining the instability status of a sample comprising:
(a) preparing a mixture comprising:
   (i) a plurality of non-extensible oligonucleotides, wherein each of the at least two non-extensible nucleotides comprises, from 5′ to 3′,
      (A) a first binding sequence that is identical to the reverse complement of at least part of an upstream sequence of a DNA template molecule;
      (B) a second binding sequence that is identical to the reverse complement of a microsatellite repetitive sequence or a variant thereof, wherein the microsatellite repetitive sequence is positioned 3′ to the upstream sequence;
      (C) a third binding sequence that is identical to the reverse complement of at least part of a downstream sequence of the DNA template molecule, wherein the downstream sequence is positioned 3′ to the microsatellite repetitive sequence; and
      (D) a terminator sequence comprising only adenine or only thymine nucleotides, and wherein the terminator sequence is not identical to the reverse complement of the at least part of the downstream sequence,
   wherein the plurality of non-extensible oligonucleotides target at least two microsatellite loci;
   (ii) the DNA template molecule, wherein the DNA template molecule is obtained from the sample;
   (iii) a DNA polymerase;
   (iv) dNTPs;
   (v) at least two primer sets, wherein the at least two primer sets are capable of amplifying the at least two microsatellite loci;
(b) subjecting the mixture to at least seven cycles of thermal cycling to produce at least one amplicon of each of the at least two microsatellite loci; and
(c) determining the instability status of the sample based on analysis of the at least one amplicon of each of the at least two microsatellite loci obtained in step (b).
   34. The method of embodiment 33, wherein the sample is a human sample.
   35. The method of embodiment 33 or 34, wherein the sample comprises cell-free DNA.
   36. The composition of any one of embodiments 1-17 for the use in the in vitro diagnosis of microsatellite instability score or microsatellite instability status of a sample or subject.
   37. The composition of embodiment 36, wherein the sample is a human sample or human subject.
   38. The kit of any one of embodiments 28-32 for the use in the in vitro diagnosis of microsatellite instability score or microsatellite instability status of a sample or subject.
   39. The kit of embodiment 38, wherein the sample is a human sample or human subject.

Having now generally described the disclosure, the same will be more readily understood through reference to the following examples that are provided by way of illustration, and are not intended to be limiting of the present disclosure, unless specified.

### EXAMPLES

### Example 1. Blocker Displacement Amplification on the NR21 Microsatellite Locus

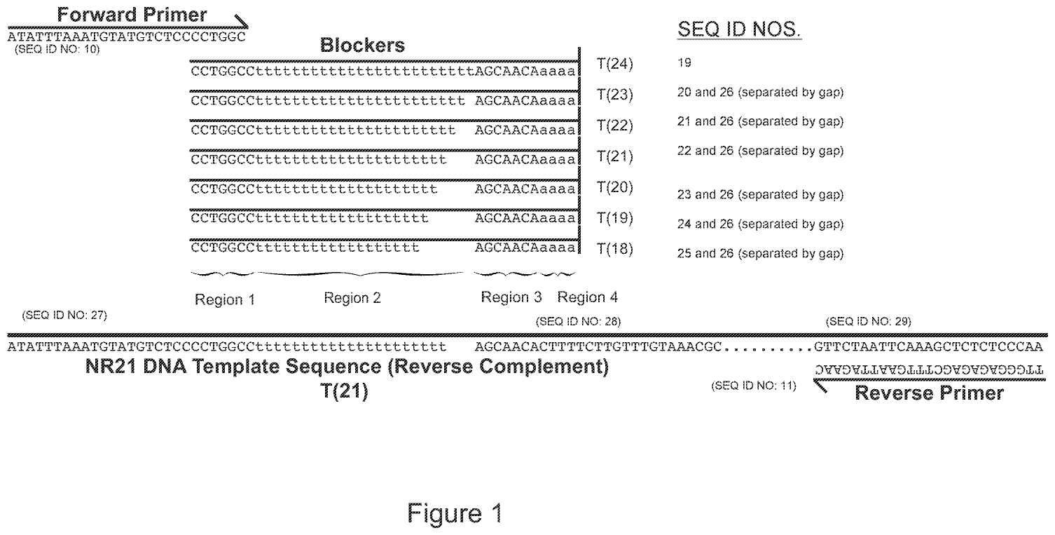

NA18537 human genomic DNA comprises a 21-adenine microsatellite repeat at the NR21 microsatellite locus. The NR21 microsatellite locus is targeted for amplification using multiple non-extensible oligonucleotides. See, for example, FIGS. 1-3. The human genomic DNA is considered a “healthy sample” DNA template for this Example.

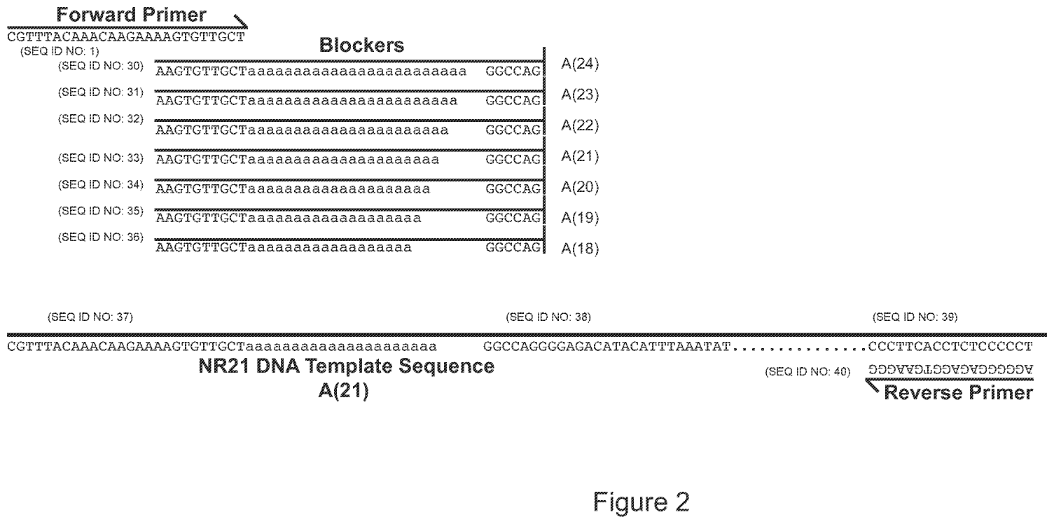

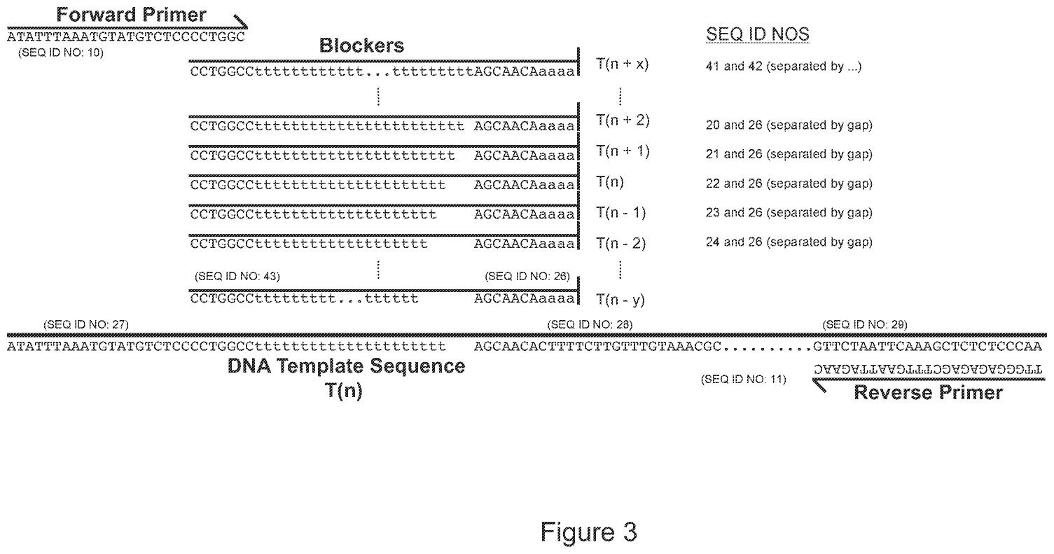

Although Example 1 pertains to detection of the NR21 microsatellite locus by amplifying and/or blocking the negative strand of the DNA template, the positive strand could be amplified and/or blocked interchangeably. See FIG. 2. It will also be appreciated that although Example 1 uses seven non-extensible oligonucleotides, fewer or greater numbers of non-extensible oligonucleotides could also be used. See FIG. 3.

A synthetic template, termed gBlock11A, is generated. The gBlock11A template comprises only 11 adenine nucleotides in the microsatellite repeat (e.g., 10 fewer adenines compared to the healthy sample).

Multiple non-extensible oligonucleotides are designed as shown in FIG. 1. Non-extendible oligonucleotides comprising 18, 19, 20, 21, 22, 23, or 24 adenines in Region 2 are prepared (the collection of the seven non-extensible oligonucleotides will be referred to as “the blockers” for the remainder of Example 1).

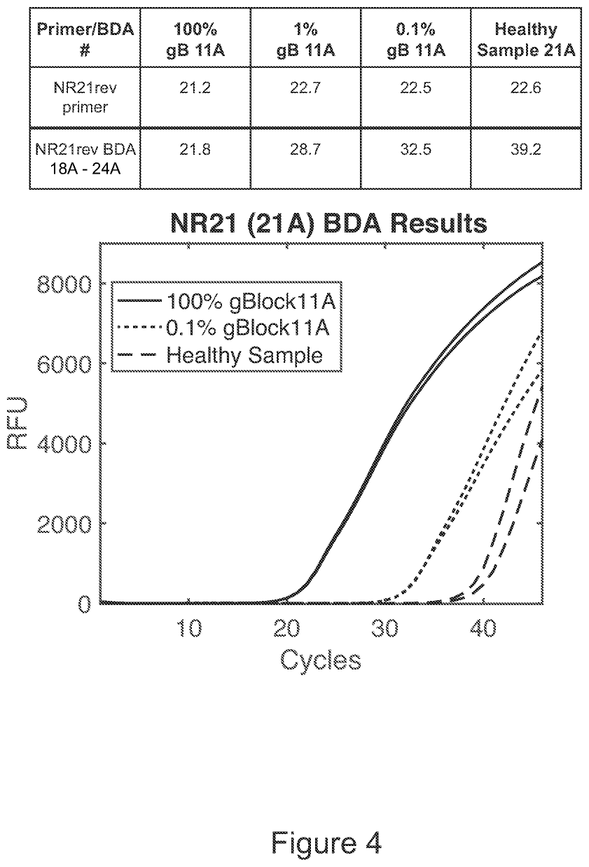

PCR was performed on the NA18537 and gBlock11A templates with a forward primer and a reverse primer, but without the use of the blockers. Both samples amplify effectively. The cycle threshold (Ct) values for both templates ranged between 21 and 23. See FIG. 4.

A second PCR was performed on the NA18537 and gBlock11 A templates using a forward primer, a reverse primer, and the blockers. With the blockers present, the gBlock11a template amplified effectively with a Ct of 21.8. However, amplification of the NA18537 template was suppressed, and the Ct value was 39.2. See FIG. 4.

Finally, two additional template mixtures were used for PCR: a first template mixture using a template mixture that comprised 99% NA18537 template and 1% gBlock11A template; and a second template mixture using a template mixture that comprised 99.9% NA18537 template and 0.1% gBlock11A template.

When the first template mixture was used with the forward and reverse primers, but no blockers, for PCR a Ct value of 22.7 was observed. See FIG. 4. When the blockers were added to the first template mixture and forward and reverse primers for PCR the Ct value dropped to 28.7. See FIG. 4. Similarly, when the second template mixture was used with the forward and reverse primers, but no blockers, for PCR a Ct value of 22.5 was observed. See FIG. 4. When the blockers were added to the second template mixture and forward and reverse primers for PCR the Ct value dropped to 32.5. See FIG. 4.

The PCR using the second template mixture, the blockers, and the forward and reverse primers has a Ct value that is 6.7 cycles earlier than the healthy sample's Ct value. Therefore, the results of the PCR using the first template mixture and second template mixture indicate that the methods and compositions provided herein have a 0.1% analytical limit of detection.

1. A kit comprising:
(a) a plurality of non-extensible oligonucleotides, wherein each of the plurality of non-extensible nucleotides comprises, from 5′ to 3′,
(i) a first binding sequence that is identical to the reverse complement of at least part of an upstream sequence of a DNA template molecule;
(ii) a second binding sequence that is identical to the reverse complement of a microsatellite repetitive sequence or a variant thereof, wherein the microsatellite repetitive sequence is positioned 3′ to the upstream sequence;
(iii) a third binding sequence that is identical to the reverse complement of at least part of a downstream sequence of the DNA template molecule, wherein the downstream sequence is positioned 3′ to the microsatellite repetitive sequence; and
(iv) a terminator sequence comprising only adenine or only thymine nucleotides, and wherein the terminator sequence is not identical to the reverse complement of the at least part of the downstream sequence; and
(b) a forward primer and a reverse primer, wherein the forward and reverse primer are capable of amplifying the DNA template molecule.

2. The kit of claim 1, wherein the kit comprises a plurality of forward primers and a plurality of reverse primers.

3. The kit of claim 1, wherein the kit comprises 5 or more forward primers and 5 or more reverse primers.

4. The kit of claim 1, wherein the kit comprises 10 or more non-extensible oligonucleotides.

5. The kit of claim 1, wherein the DNA template molecule is a human DNA template molecule.

6. A composition comprising a plurality of non-extensible oligonucleotides, wherein each of the plurality of non-extensible nucleotides comprises, from 5′ to 3′:
(a) a first binding sequence that is identical to the reverse complement of at least part of the upstream sequence;
(b) a second binding sequence that is identical to the reverse complement of the microsatellite repetitive sequence or a variant thereof;
(c) a third binding sequence that is identical to the reverse complement of at least part of the downstream sequence; and
(d) a terminator sequence comprising only adenine or only thymine nucleotides, and wherein the terminator sequence is not identical to the reverse complement of the at least part of the downstream sequence.

7. The composition of claim 6, wherein the composition further comprises: a DNA template molecule comprising, continuously from 5′ to 3′:
(a) an upstream sequence upstream to a microsatellite repetitive sequence;
(b) the microsatellite repetitive sequence; and
(c) a downstream sequence downstream to the microsatellite repetitive sequence.

8. The composition of claim 6, wherein the composition further comprises a DNA polymerase.

9. The composition of claim 8, wherein the at least one DNA polymerase is selected from the group consisting of phi29 DNA polymerase, DNA polymerase 1, large (Klenow) fragment, Klenow fragment, Bst DNA polymerase, T4 DNA polymerase, T7 DNA polymerase, Taq polymerase, Phusion® polymerase, Q5® polymerase, KAPA HiFi polymerase, Vent® DNA polymerase, LongAmp® Taq DNA polymerase, and OneTaq® DNA polymerase.

10. The composition of any claim 8, wherein the composition further comprises one or more reagents necessary for DNA polymerase activity.

11. The composition of claim 6, wherein the plurality of non-extensible oligonucleotides comprises non-identical second binding sequences.

12. The composition of claim 6, wherein the plurality of non-extensible oligonucleotides comprises identical first binding sequences and identical third binding sequences.

13. The composition of claim 6, wherein the microsatellite repetitive sequence comprises homopolymer repeats.

14. The composition of claim 6, wherein the microsatellite repetitive sequence comprises dinucleotide repeats.

15. The composition of claim 6, wherein the microsatellite repetitive sequence comprises trinucleotide repeats.

16. The composition of claim 6, wherein the microsatellite repetitive sequence comprises tetranucleotide repeats.

17. The composition of claim 6, wherein the terminator sequence comprises between 4 nucleotides and 100 nucleotides.

18. The composition of claim 6, wherein the composition further comprises a forward primer and a reverse primer.

19. The composition of claim 18, wherein the forward primer and the plurality of non-extensible oligonucleotides comprise an overlapping sequence.

20. The composition of claim 19, wherein the overlapping sequence comprises between 2 nucleotides and 15 nucleotides.

21. The composition of claim 6, wherein the plurality of non-extensible oligonucleotides comprises between 2 and 100 non-extensible oligonucleotides.

22. A method of determining the instability status of a sample comprising:
(a) preparing a mixture comprising:
(i) a plurality of non-extensible oligonucleotides, wherein each of the at least two non-extensible nucleotides comprises, from 5′ to 3′,
(A) a first binding sequence that is identical to the reverse complement of at least part of an upstream sequence of a DNA template molecule;
(B) a second binding sequence that is identical to the reverse complement of a microsatellite repetitive sequence or a variant thereof, wherein the microsatellite repetitive sequence is positioned 3′ to the upstream sequence;
(C) a third binding sequence that is identical to the reverse complement of at least part of a downstream sequence of the DNA template molecule, wherein the downstream sequence is positioned 3′ to the microsatellite repetitive sequence; and
(D) a terminator sequence comprising only adenine or only thymine nucleotides, and wherein the terminator sequence is not identical to the reverse complement of the at least part of the downstream sequence,
wherein the plurality of non-extensible oligonucleotides target at least two microsatellite loci;
(ii) the DNA template molecule, wherein the DNA template molecule is obtained from the sample;
(iii) a DNA polymerase;
(iv) dNTPs;
(v) at least two primer sets, wherein the at least two primer sets are capable of amplifying the at least two microsatellite loci;
(b) subjecting the mixture to at least seven cycles of thermal cycling to produce at least one amplicon of each of the at least two microsatellite loci; and
(c) determining the instability status of the sample based on analysis of the at least one amplicon of each of the at least two microsatellite loci obtained in step (b).

23. The method of claim 22, wherein the sample is a human sample.

24. The method of claim 22, wherein the sample comprises cell-free DNA.


SEQUENCE LISTING (43 sequences)

SEQ ID NO: 1 - DNA, 26 bp, Artificial Sequence
  Description of Artificial Sequence Synthetic polynucleotide
cgtttacaaacaagaaaagtgttgct

SEQ ID NO: 2 - DNA, 25 bp, Artificial Sequence
  Description of Artificial Sequence Synthetic polynucleotide
tgttttattagggggagaggtgaag

SEQ ID NO: 3 - DNA, 41 bp, Artificial Sequence
  Description of Artificial Sequence Synthetic polynucleotide
aagtgttgctaaaaaaaaaaaaaaaaaaaaaggccagaaat

SEQ ID NO: 4 - DNA, 40 bp, Artificial Sequence
  Description of Artificial Sequence Synthetic polynucleotide
aagtgttgctaaaaaaaaaaaaaaaaaaaaggccagaaat

SEQ ID NO: 5 - DNA, 39 bp, Artificial Sequence
  Description of Artificial Sequence Synthetic polynucleotide
agtgttgctaaaaaaaaaaaaaaaaaaaggccaggaata

SEQ ID NO: 6 - DNA, 41 bp, Artificial Sequence
  Description of Artificial Sequence Synthetic polynucleotide
aagtgttgctaaaaaaaaaaaaaaaaaaaaaaggccaaaaa

SEQ ID NO: 7 - DNA, 42 bp, Artificial Sequence
  Description of Artificial Sequence Synthetic polynucleotide
aagtgttgctaaaaaaaaaaaaaaaaaaaaaaaggccaaaaa

SEQ ID NO: 8 - DNA, 38 bp, Artificial Sequence
  Description of Artificial Sequence Synthetic polynucleotide
agtgttgctaaaaaaaaaaaaaaaaaaggccaggaata

SEQ ID NO: 9 - DNA, 43 bp, Artificial Sequence
  Description of Artificial Sequence Synthetic polynucleotide
aagtgttgctaaaaaaaaaaaaaaaaaaaaaaaaggccaaaaa

SEQ ID NO: 10 - DNA, 26 bp, Artificial Sequence
  Description of Artificial Sequence Synthetic polynucleotide
atatttaaatgtatgtctcccctggc

SEQ ID NO: 11 - DNA, 25 bp, Artificial Sequence
  Description of Artificial Sequence Synthetic polynucleotide
ttgggagagagctttgaattagaac

SEQ ID NO: 12 - DNA, 39 bp, Artificial Sequence
  Description of Artificial Sequence Synthetic polynucleotide
cctggcctttttttttttttttttttttagcaacaaaaa

SEQ ID NO: 13 - DNA, 38 bp, Artificial Sequence
  Description of Artificial Sequence Synthetic polynucleotide
cctggccttttttttttttttttttttagcaacaaaaa

SEQ ID NO: 14 - DNA, 38 bp, Artificial Sequence
  Description of Artificial Sequence Synthetic polynucleotide
cctggcctttttttttttttttttttagcaacacaaaa

SEQ ID NO: 15 - DNA, 39 bp, Artificial Sequence
  Description of Artificial Sequence Synthetic polynucleotide
cctggccttttttttttttttttttttttagcaactaaa

SEQ ID NO: 16 - DNA, 40 bp, Artificial Sequence
  Description of Artificial Sequence Synthetic polynucleotide
cctggcctttttttttttttttttttttttagcaactaaa

SEQ ID NO: 17 - DNA, 37 bp, Artificial Sequence
  Description of Artificial Sequence Synthetic polynucleotide
cctggccttttttttttttttttttagcaacacaaaa

SEQ ID NO: 18 - DNA, 41 bp, Artificial Sequence
  Description of Artificial Sequence Synthetic polynucleotide
cctggccttttttttttttttttttttttttagcaactaaa

SEQ ID NO: 19 - DNA, 42 bp, Artificial Sequence
  Description of Artificial Sequence Synthetic polynucleotide
cctggccttttttttttttttttttttttttagcaacaaaaa

SEQ ID NO: 20 - DNA, 30 bp, Artificial Sequence
  Description of Artificial Sequence Synthetic polynucleotide
cctggccttttttttttttttttttttttt

SEQ ID NO: 21 - DNA, 29 bp, Artificial Sequence
  Description of Artificial Sequence Synthetic polynucleotide
cctggcctttttttttttttttttttttt

SEQ ID NO: 22 - DNA, 28 bp, Artificial Sequence
  Description of Artificial Sequence Synthetic polynucleotide
cctggccttttttttttttttttttttt

SEQ ID NO: 23 - DNA, 27 bp, Artificial Sequence
  Description of Artificial Sequence Synthetic polynucleotide
cctggcctttttttttttttttttttt

SEQ ID NO: 24 - DNA, 26 bp, Artificial Sequence
  Description of Artificial Sequence Synthetic polynucleotide
cctggccttttttttttttttttttt

SEQ ID NO: 25 - DNA, 25 bp, Artificial Sequence
  Description of Artificial Sequence Synthetic polynucleotide
cctggcctttttttttttttttttt

SEQ ID NO: 26 - DNA, 11 bp, Artificial Sequence
  Description of Artificial Sequence Synthetic polynucleotide
agcaacaaaaa

SEQ ID NO: 27 - DNA, 48 bp, Artificial Sequence
  Description of Artificial Sequence Synthetic polynucleotide
atatttaaatgtatgtctcccctggccttttttttttttttttttttt

SEQ ID NO: 28 - DNA, 27 bp, Artificial Sequence
  Description of Artificial Sequence Synthetic polynucleotide
agcaacacttttcttgtttgtaaacgc

SEQ ID NO: 29 - DNA, 25 bp, Artificial Sequence
  Description of Artificial Sequence Synthetic polynucleotide
gttctaattcaaagctctctcccaa

SEQ ID NO: 30 - DNA, 34 bp, Artificial Sequence
  Description of Artificial Sequence Synthetic polynucleotide
aagtgttgctaaaaaaaaaaaaaaaaaaaaaaaa

SEQ ID NO: 31 - DNA, 33 bp, Artificial Sequence
  Description of Artificial Sequence Synthetic polynucleotide
aagtgttgctaaaaaaaaaaaaaaaaaaaaaaa

SEQ ID NO: 32 - DNA, 32 bp, Artificial Sequence
  Description of Artificial Sequence Synthetic polynucleotide
aagtgttgctaaaaaaaaaaaaaaaaaaaaaa

SEQ ID NO: 33 - DNA, 31 bp, Artificial Sequence
  Description of Artificial Sequence Synthetic polynucleotide
aagtgttgctaaaaaaaaaaaaaaaaaaaaa

SEQ ID NO: 34 - DNA, 30 bp, Artificial Sequence
  Description of Artificial Sequence Synthetic polynucleotide
aagtgttgctaaaaaaaaaaaaaaaaaaaa

SEQ ID NO: 35 - DNA, 29 bp, Artificial Sequence
  Description of Artificial Sequence Synthetic polynucleotide
aagtgttgctaaaaaaaaaaaaaaaaaaa

SEQ ID NO: 36 - DNA, 28 bp, Artificial Sequence
  Description of Artificial Sequence Synthetic polynucleotide
aagtgttgctaaaaaaaaaaaaaaaaaa

SEQ ID NO: 37 - DNA, 47 bp, Artificial Sequence
  Description of Artificial Sequence Synthetic polynucleotide
cgtttacaaacaagaaaagtgttgctaaaaaaaaaaaaaaaaaaaaa

SEQ ID NO: 38 - DNA, 27 bp, Artificial Sequence
  Description of Artificial Sequence Synthetic polynucleotide
ggccaggggagacatacatttaaatat

SEQ ID NO: 39 - DNA, 18 bp, Artificial Sequence
  Description of Artificial Sequence Synthetic polynucleotide
cccttcacctctccccct

SEQ ID NO: 40 - DNA, 18 bp, Artificial Sequence
  Description of Artificial Sequence Synthetic polynucleotide
agggggagaggtgaaggg

SEQ ID NO: 41 - DNA, 19 bp, Artificial Sequence
  Description of Artificial Sequence Synthetic polynucleotide
cctggcctttttttttttt

SEQ ID NO: 42 - DNA, 20 bp, Artificial Sequence
  Description of Artificial Sequence Synthetic polynucleotide
tttttttttagcaacaaaaa

SEQ ID NO: 43 - DNA, 16 bp, Artificial Sequence
  Description of Artificial Sequence Synthetic polynucleotide
cctggccttttttttt